(19) United States
(12) Patent Application Publication
(10) Pub. No.: US 2023/0256248 A1
(43) Pub. Date: Aug. 17, 2023

(54) DEEP BRAIN STIMULATION

(71) Applicant: Oxford University Innovation Limited, Oxford (GB)

(72) Inventors: Gihan WEERASINGHE, Oxford (Oxfordshire) (GB); Rafal BOGACZ, Oxford (Oxfordshire) (GB)

(21) Appl. No.: 18/018,072

(22) PCT Filed: Aug. 6, 2021
(86) PCT No.: PCT/GB2021/052039

§ 371 Date: Jan. 26, 2023

(30) Foreign Application Priority Data
Aug. 7, 2020 (GB) 2012308.9

(51) Int. Cl. A61N 1/36

(52) U.S. Cl. CPC A61N 1/3606; A61N 1/36171; A61N 1/36139; A61N 1/36067; A61N 1/0534

(57) ABSTRACT

There is provided a method of generating deep brain stimulation signals, the method comprising receiving a plurality of sensor signals from a corresponding plurality of sensors on or in a subject, and using the received sensor signals to generate a plurality of stimulation signals for application at a corresponding plurality of target sites in the brain of the subject. There is further provided a method of generating stimulation signals, the method comprising receiving a plurality of sensor signals from a corresponding plurality of sensors on or in a subject, and using the received sensor signals to generate a plurality of stimulation signals for application at a corresponding plurality of target sites on or in the subject using a model of the response of neurons in the subject to the stimulation signals that models neural tissue as a plurality of coupled populations of neurons.

The invention relates to the generation of signals for stimulation of a subject. Typically, the stimulation is used to stimulate a target site in the nervous system (e.g. the brain) of a human or animal subject.

Deep brain stimulation (DBS) is a well-established and effective treatment option for a variety of neurological disorders, including Parkinson's disease (PD) and essential tremor (ET). DBS involves delivering stimulation through electrodes implanted deep into the brain and targeting regions thought to be implicated in the disease, which in the case of PD is typically the subthalamic nucleus (STN) and for ET the ventral intermediate nucleus (VIM). PD is a common movement disorder caused by the death of dopaminergic neurons in the substantia nigra. Primarily, symptoms manifest as slowness of movement (bradykinesia), muscle stiffness (rigidity) and tremor. ET is purportedly the most common movement disorder, affecting just under 1% of the world population [1] with the main symptom being involuntary shaking most commonly in the upper limbs. Despite its prevalence, the pathophysiology of ET remains elusive, although the cortex, thalamus and cerebellum are all thought to be involved in the disease [1].

Symptoms of these disorders are thought to be due to overly synchronous activity within neural populations. For PD patients, higher power in the beta frequency range (13-30 Hz) of the local field potential (LFP) measured in the STN has been shown to correlate with motor impairment [2] while thalamic activity in ET patients is strongly correlated with tremor measured using the wrist flexor EMG [3]. It is thought that DBS acts to desynchronise this pathological activity leading to a reduction in the symptom severity.

A typical DBS system consists of a lead, an implantable pulse generator (IPG) and a unit to be operated by the patient. The DBS lead terminates with an electrode, which is typically divided into multiple contacts. Post-surgery, clinicians manually tune the various parameters of stimulation, such as the frequency, amplitude and pulse width, in an attempt to achieve optimal therapeutic benefit. The choice of stimulation frequency in particular is known to be crucial for efficacy with high frequency (HF) DBS (120-180 Hz) being found to be effective for both PD and ET patients [4].

Despite the effectiveness of conventional HFDBS in treating PD and ET, it is believed that improvements to the efficiency and efficacy can be achieved by using more elaborate stimulation patterns informed by mathematical models. Coordinated reset (CR) neuromodulation is an open-loop DBS strategy where brief HF pulse trains are applied through different contacts of a stimulation electrode [5]. The efficacy of CR was first demonstrated theoretically, where precisely-timed delivery of HF pulses can be shown to desynchronise a system of coupled oscillators [5]. In practice, CR has been shown to yield both acute and long-lasting benefits in nonhuman primates.

Multi-contact electrodes powered by independent current sources are a recent development in DBS technology. IPGs with multiple independent current sources are the ‘cutting-edge’ of DBS technology which, unlike their single current source counterparts, allow for current to be delivered independently to each contact. This gives increased control and flexibility over the shape of the electric fields delivered through the electrodes, allowing for more precise targeting of pathological regions and the possibility of delivering more complex potential fields over space, in addition to allowing for the possibility of recording activity from different regions. The use of multiple contacts for DBS, however, naturally leads to increased complexity, as many more stimulation strategies are now possible. This has created the need to better understand how applying DBS through multiple contacts can affect the treatment. In order to realise the potential of such systems, algorithms must be developed to deal with their increased complexity, and address the question of how to best apply DBS to multiple regions to maximally desynchronise brain activity.

Therefore, it is an object of the invention to provide improved multi-electrode stimulation systems.

According to an aspect of the invention, there is provided a method of generating deep brain stimulation signals, the method comprising receiving a plurality of sensor signals from a corresponding plurality of sensors on or in a subject, and using the received sensor signals to generate a plurality of stimulation signals for application at a corresponding plurality of target sites in the brain of the subject.

In conventional DBS systems, following tuning of parameters by a clinician, stimulation is then provided constantly, or ‘open-loop’, according to these parameters. The efficacy, efficiency and side-effects of the treatment can be improved by stimulating ‘closed-loop’, according to the symptoms of a patient. Closed-loop stimulation aims to deliver stimulation on the basis of feedback from a patient or subject. There is a growing body of evidence [6, 7, 2] suggesting that closed-loop stimulation has the potential to offer improvements in terms of efficacy, efficiency and reduction in side effects. Applying such closed-loop control to multi-electrode systems that target a plurality of target sites using a plurality of stimulation signals has the potential to provide even greater benefit.

According to another aspect of the invention, there is provided a method of generating stimulation signals, the method comprising receiving a plurality of sensor signals from a corresponding plurality of sensors on or in a subject, using the received sensor signals to generate a plurality of stimulation signals for application at a corresponding plurality of target sites on or in the subject using a model of the response of neurons in the subject to the stimulation signals that models neural tissue as a plurality of coupled populations of neurons.

The greater complexity of multi-electrode systems means that there is a need to understand how applying DBS to multiple regions (or neural populations) can affect the efficacy and efficiency of the treatment. By modelling the response of neurons using a plurality of coupled populations, it is possible to derive analytical expressions which predict how symptom severity should change as a result of applying stimulation. On the basis of these expressions, it is possible to derive an algorithm describing when the stimulation should be delivered to individual target sites.

In some embodiments, generating the plurality of stimulation signals comprises determining a population activity for each population of neurons and determining the amplitude and phase of each population activity, the population activity of each population being a measure of neural activity among neurons in that population. Depending on the response of different classes of neurons, it can be advantageous to generate signals based on the activity of individual populations. Determining population activities allows the determination of the conditions for when stimulation using information from individual contacts is likely to be advantageous.

In some embodiments, determining the population activities comprises applying independent component analysis to the sensor signals. Independent component analysis is a computationally efficient way to identify the contributions to a signal from different sources.

In some embodiments, generating the plurality of stimulation signals further comprises determining a composite signal using a weighted combination of the population activities, and determining the amplitude and phase of the composite signal. Using a composite signal determined using a combination of population activities allows for characterisation of an overall activity of neurons without having to measure a separate signal to that used to determine the population activities.

In some embodiments, generating the plurality of stimulation signals further comprises, for each of a plurality of time steps, choosing the plurality of stimulation signals to maximally reduce the amplitude of the composite signal over the time step. This provides a convenient optimisation problem to determine the signals that should be applied to each target site to lower the overall severity of symptoms based on the combination of population activities. In some embodiments, maximally reducing the amplitude of the composite signal comprises, for each time step, calculating the rate of change in amplitude of the composite signal based on the composite signal and the plurality of population activities. Basing the calculation on both the composite signal representing overall activity in the region and the individual population activities can be advantageous, particularly for some types of neural tissue.

In some embodiments, the plurality of stimulation signals are chosen subject to a constraint on the total charge density within a region of the subject, and/or a constraint on the charge applied by each electrode. These constraints prevent the electrodes applying too great a charge either individually or collectively that could cause undesirable side effects.

In some embodiments, the weights in the weighted combination are such that the amplitude of the composite signal is correlated to (preferably proportional to) a measure of severity of a symptom of the subject. This ensures that the composite signal is representative of symptom severity such that use of the composite signal will allow more effective determination of the stimulation signals that should be applied to each target site.

In some embodiments, the model models each population of neurons as a plurality of coupled oscillators. The electrical activity of neurons typically has a periodic behaviour, and can affect the activity of neighbouring neurons. Therefore, coupled oscillators make a good approximation to their behaviour. Preferably, the coupled oscillators comprise Kuramoto oscillators. The Kuramoto model is a known model for neural activity that provides a good approximation to the behaviour of groups of neurons.

In some embodiments, the model models the response of the neurons to the stimulation signals as being dependent on the phase of oscillations of the neurons. Some neurons tend to respond to external stimulation uniformly regardless of where they are in their cycle of activity, while other neurons have a response that depends on the phase of their oscillations. Taking account of this will improve the outcome of stimulation by more accurately determining the effect a particular stimulation will have on the target neurons.

In some embodiments, the plurality of sensor signals represent electrical activity in the subject. Direct measurement of electrical activity is an effective way to measure neural activity and thereby determine the parameters of the plurality of populations of neurons.

In some embodiments, the electrical activity is a local field potential produced by neurons in a region of the brain of the subject. Measuring a local field potential, rather than for example a potential from a single neuron or a small number of neurons, means that larger electrodes can be used, which are easier to handle and implant.

In some embodiments, the sensors are inertial sensors, and the plurality of sensor signals represent movement of the subject. Inertial sensors can provide more direct measurement of symptoms that are to be treated by the stimulation, thereby giving a more accurate picture of the effectiveness of the stimulation.

In some embodiments, the stimulation signals comprise a plurality of pulses. Pulsed signals provide a convenient way to vary the stimulation intensity.

In some embodiments, the stimulation signals have a carrier frequency of at least 20 Hz and/or at most 250 Hz. This range of frequencies has been found to be particularly effective in relieving symptoms using pulsed stimulation signals.

In some embodiments, the application of the stimulation signals is used for treatment of Parkinson's disease, epilepsy, obsessive compulsive disorder, and/or essential tremor. These conditions are particularly responsive to stimulation signals of the type generated by the method.

According to another aspect of the invention, there is provided a system for applying stimulation signals, the system comprising a processor configured to generate a plurality of stimulation signals according to the method of any one of the preceding claims, and an electrical circuit configured to provide the plurality of stimulation signals to a corresponding plurality of electrodes for application at the plurality of target sites. This system allows for the provision of stimulation signals with the benefits of the method as described above.

In some embodiments, the electrical circuit comprises the plurality of electrodes. This may be convenient as it allows the properties of the electrodes to be tailored to the electrical circuit to more effectively deliver the stimulation signals.

In some embodiments, the plurality of electrodes are configured to be implanted into the brain of the subject. This provides the most direct stimulation for treatment of neurological conditions.

In some embodiments, the system further comprises a plurality of sensors configured to be placed on or in the subject, the sensors configured to generate the plurality of sensor signals, and transmit the sensor signals to the processor. Including the sensors in the system allows their properties to be chosen and accounted for in the design of the system.

In some embodiments, the sensors are configured to be implanted into the brain of the subject, and the plurality of sensor signals represent electrical activity in the brain of the subject. Direct measurement of electrical activity in the brain can be used to give an accurate indication of neural activity.

In some embodiments, the plurality of sensors are the plurality of electrodes. Combining the functionality of the electrodes and sensors means that fewer components are needed, and the sensor signals directly reflect the electrical activity in the target sites to which the stimulation signals are applied.

Embodiments of the invention will now be described, by way of example only, with reference to the accompanying drawings in which corresponding reference symbols represent corresponding parts, and in which:

FIG. 1 is a schematic diagram of a system for applying stimulation signals;

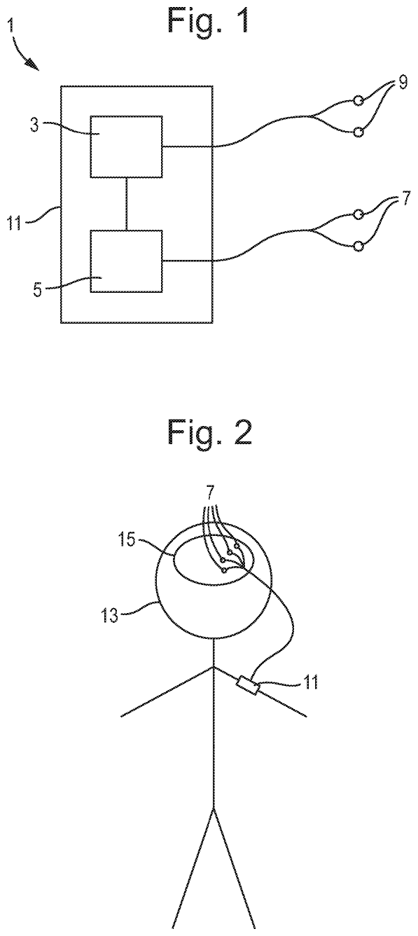

FIG. 2 is a schematic of the system of FIG. 1 attached to a subject;

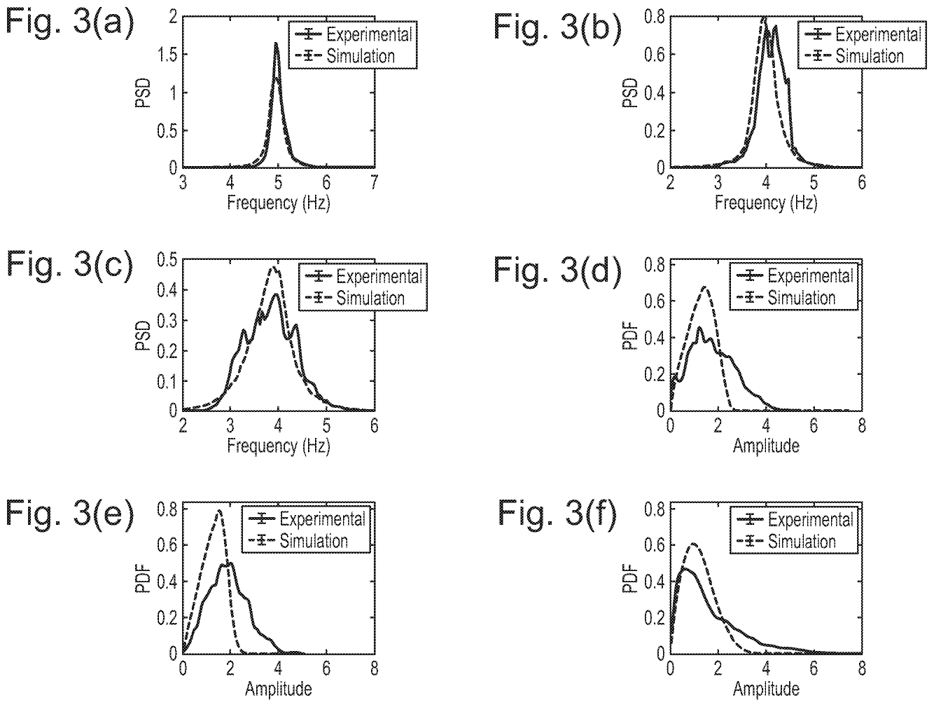

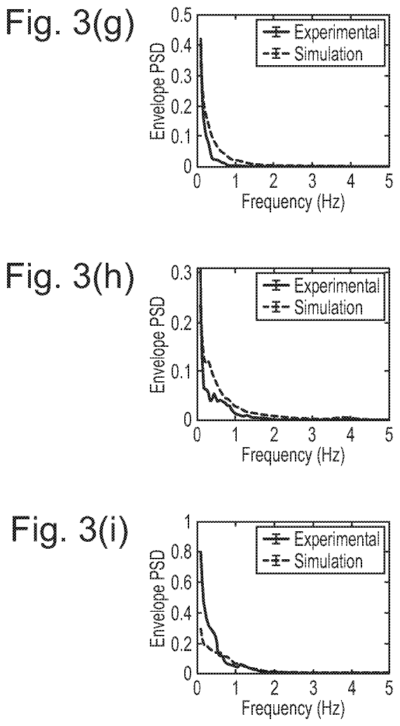

FIG. 3 shows fits of a Kuramoto model to various features extracted from tremor data from ET patients;

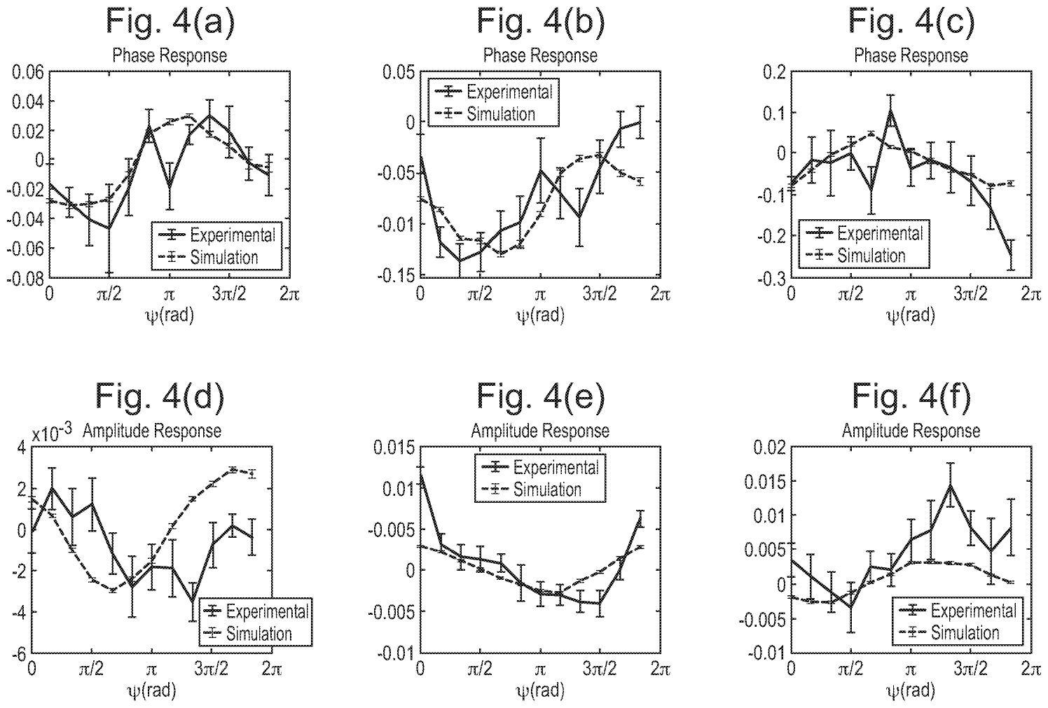

FIG. 4 is a comparison of averaged phase and amplitude response curves between experimental and fitted data from the Kuramoto model;

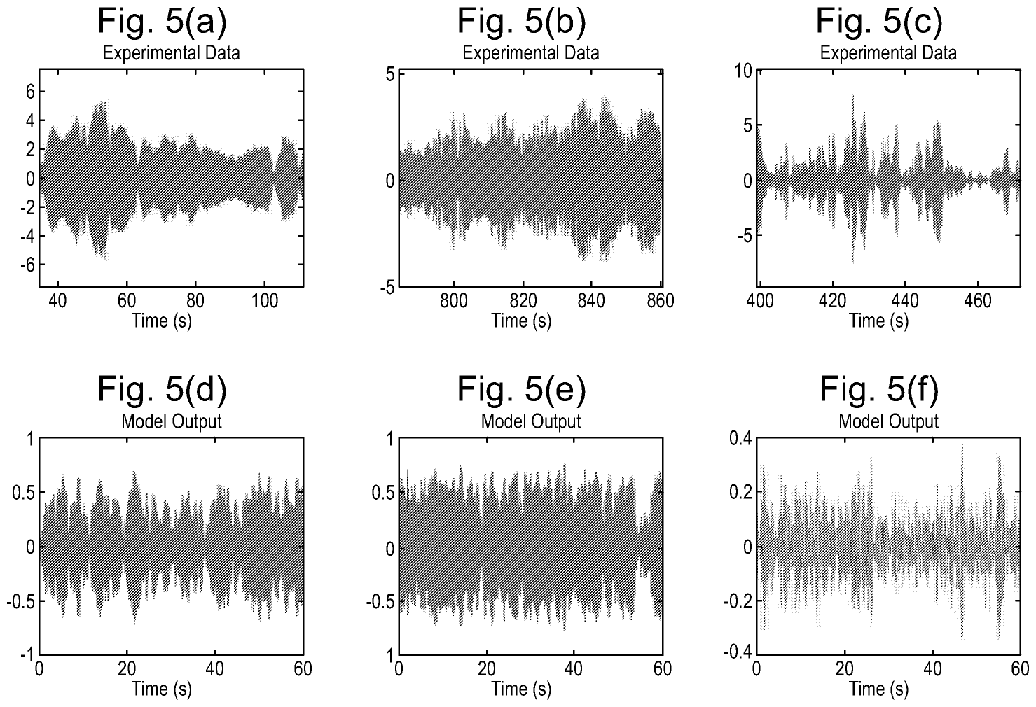

FIG. 5 is a comparison of experimentally measured tremor data and the output of the Kuramoto model in the time domain;

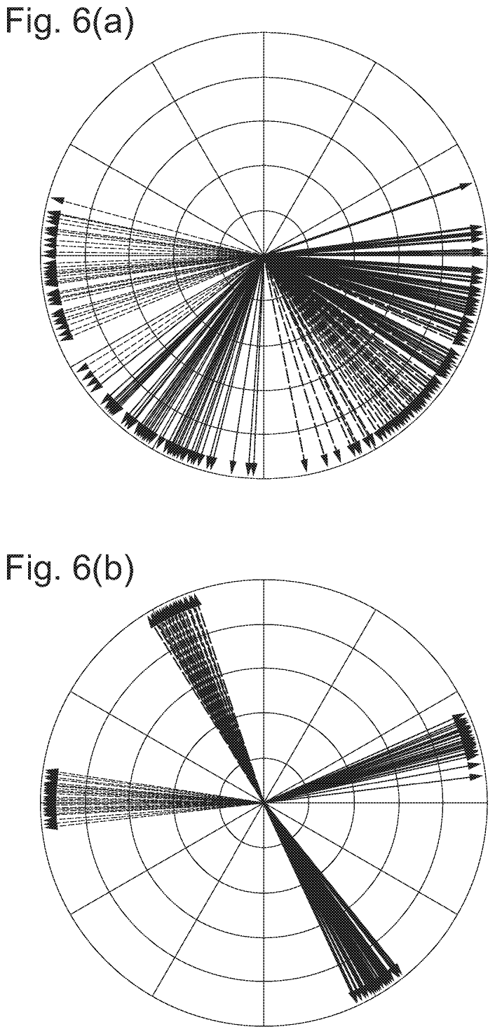

FIG. 6 shows different distributions of oscillators in phase space;

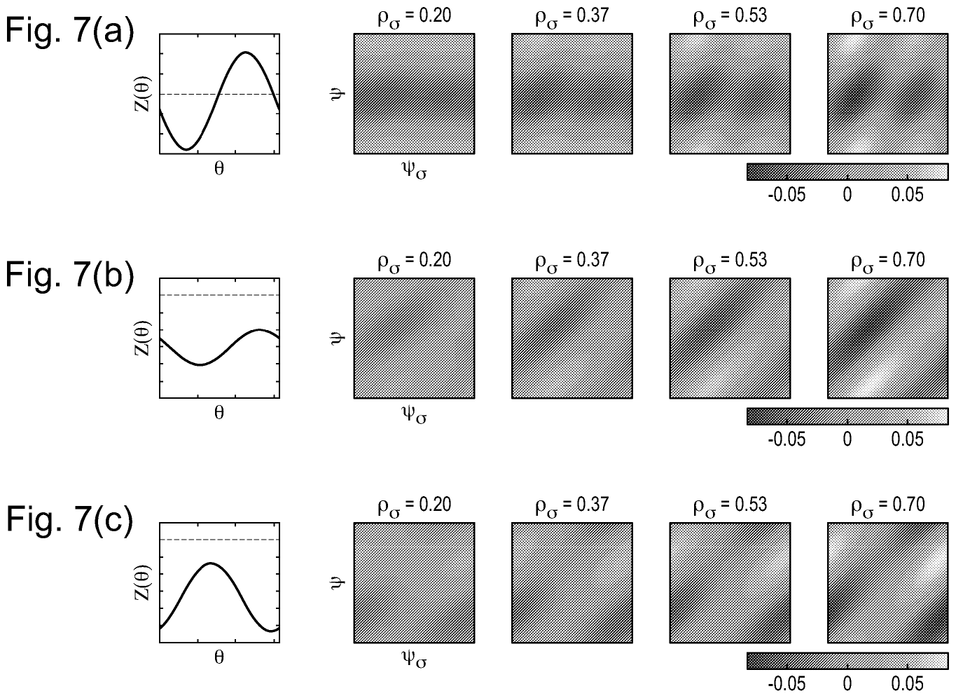

FIG. 7 shows the contribution of a single population to the global amplitude response at different local amplitudes and for different neuronal phase response curves;

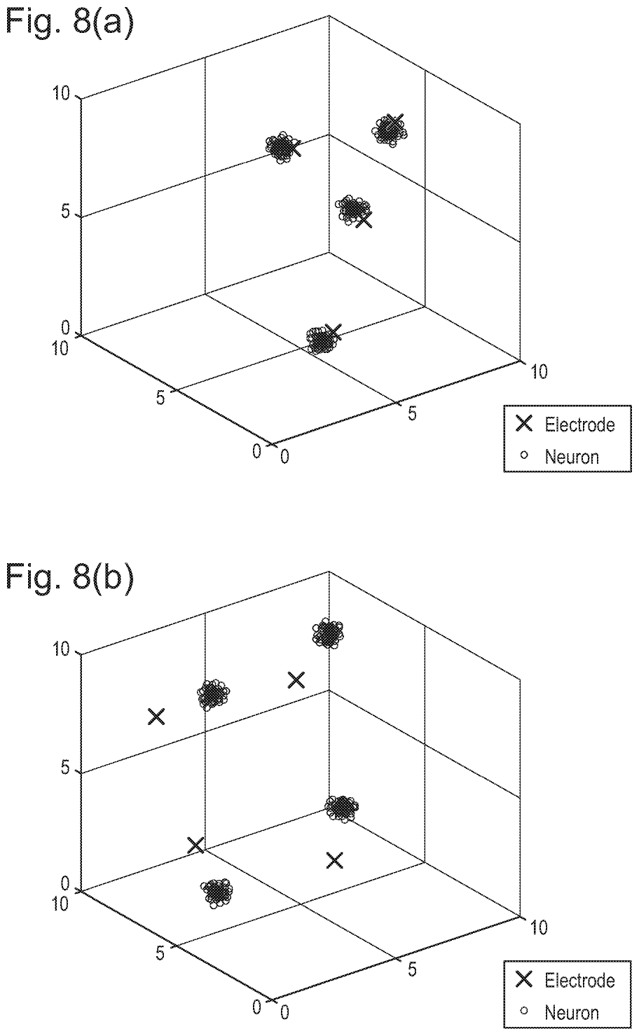

FIG. 8 shows visualisations of the relative positioning of neuronal populations and electrodes;

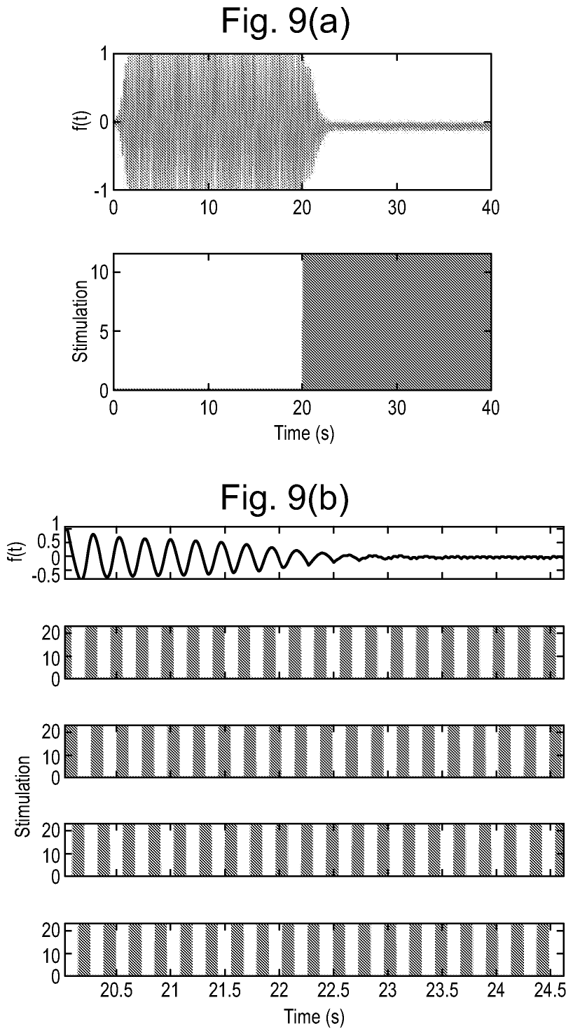

FIG. 9 shows simulations of the effect of coordinated reset stimulation on symptom severity;

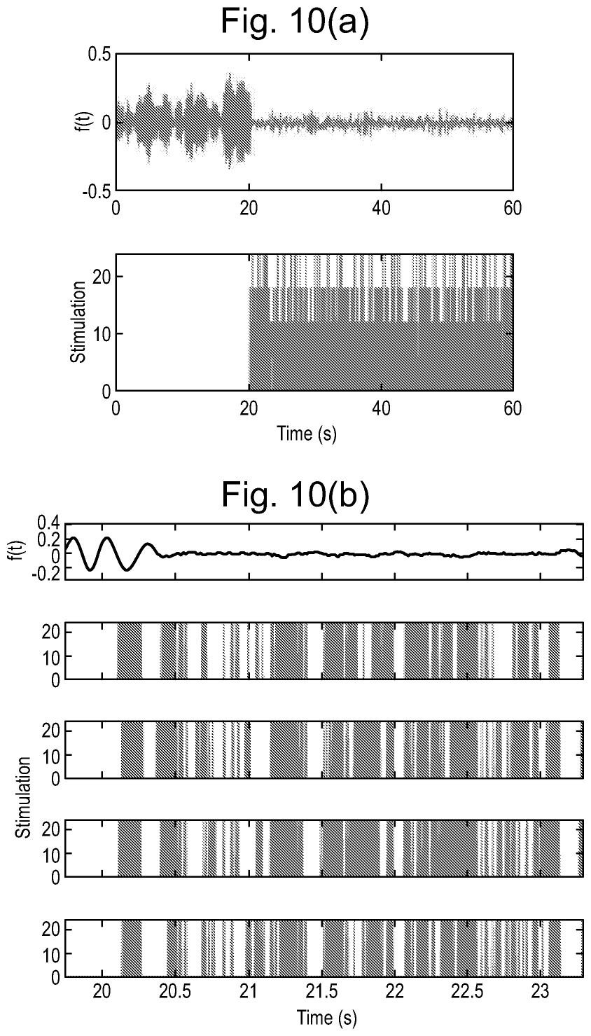

FIG. 10 shows simulations of the effect of the present stimulation method on symptom severity;

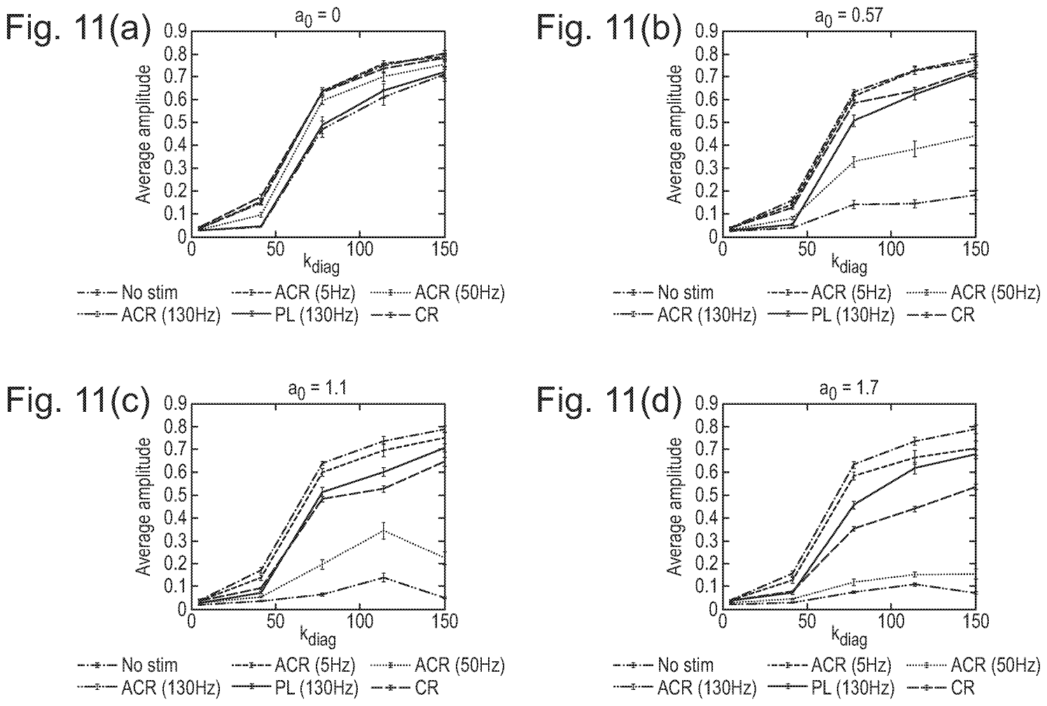

FIG. 11 shows the average amplitude of a simulated Kuramoto system for different stimulation strategies;

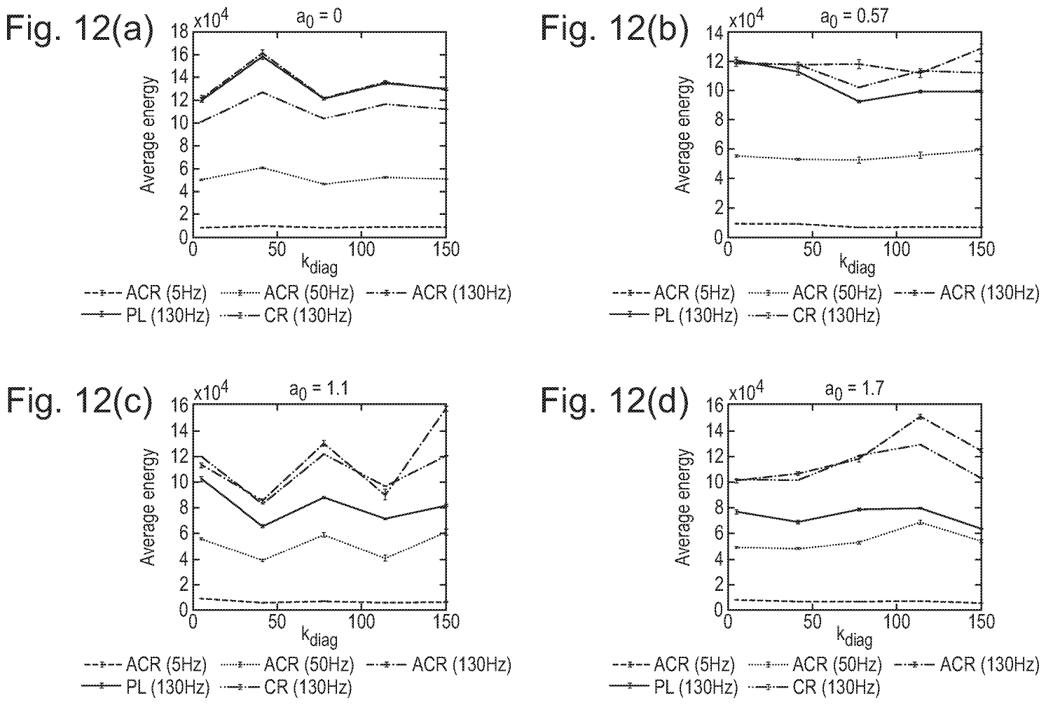

FIG. 12 shows the average energy used by different stimulation strategies;

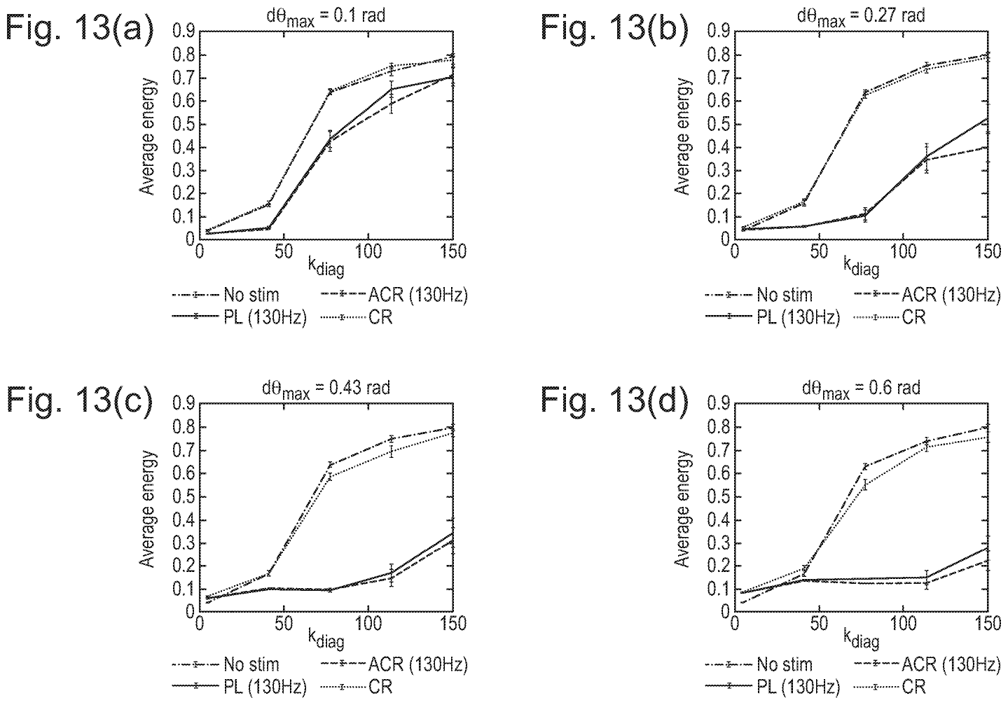

FIG. 13 shows the average amplitude of a simulated Kuramoto system for different stimulation strategies in a system where the amplitude response depends on local neuronal population activity;

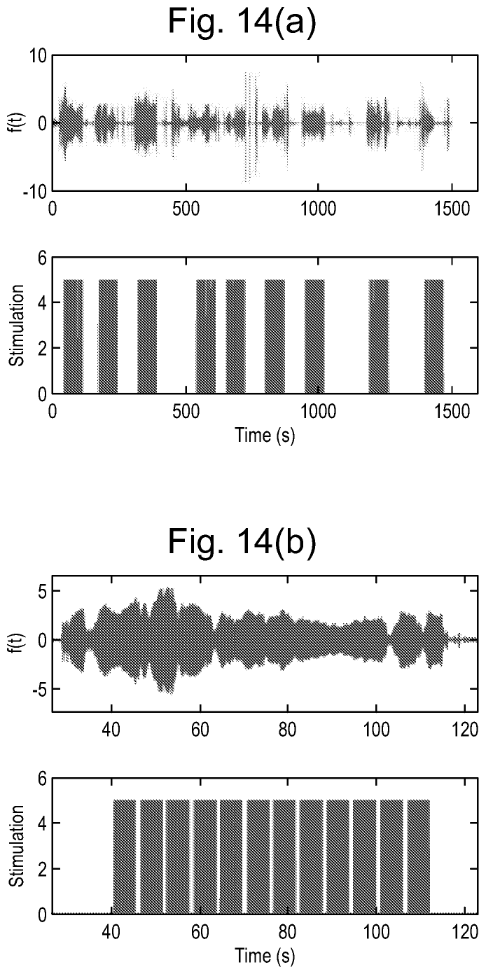

FIG. 14 shows tremor data for one of the patients from the dataset used for the analysis discussed further below;

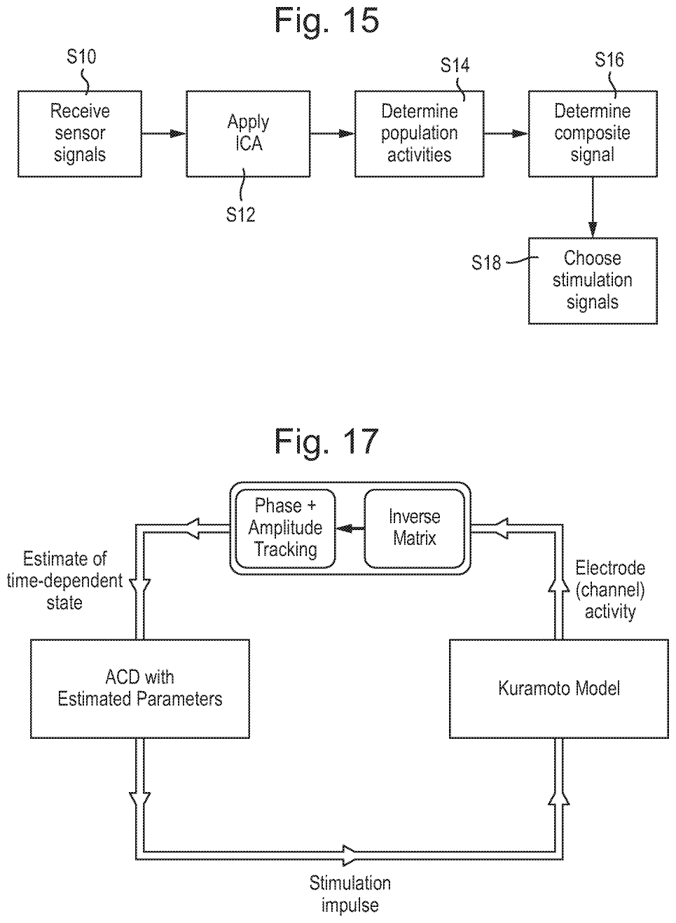

FIG. 15 shows a flowchart of an embodiment of the method;

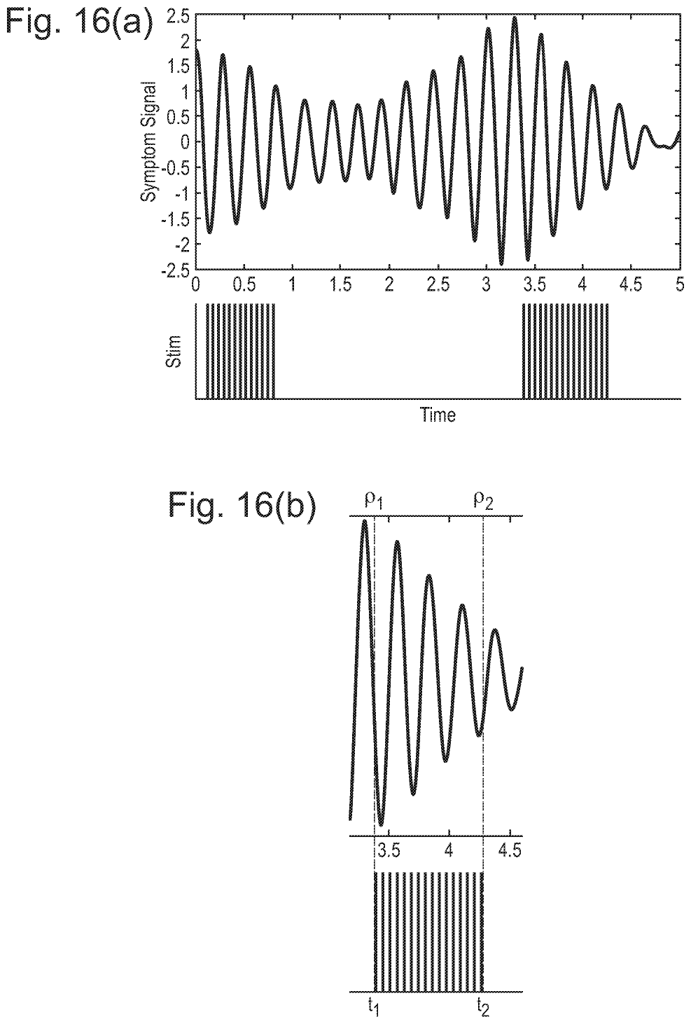

FIG. 16 shows signals used for calibration of the method to determine response parameters;

FIG. 17 is a flowchart visualising simulations used in further testing of the method;

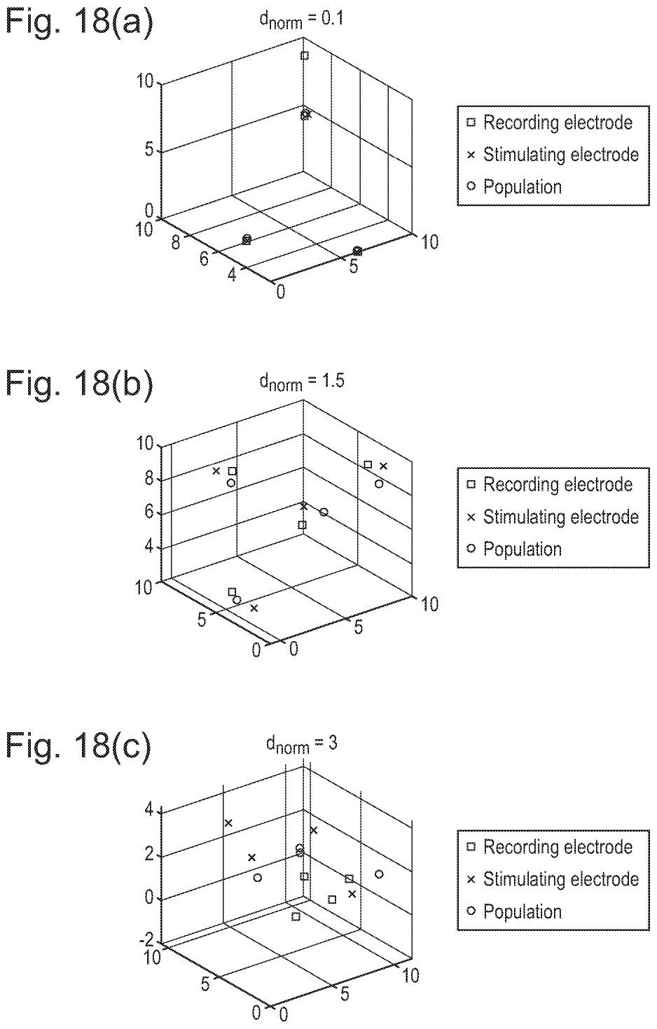

FIG. 18 shows visualisations of the relative positioning of neural populations, electrodes, and sensors in further testing of the method;

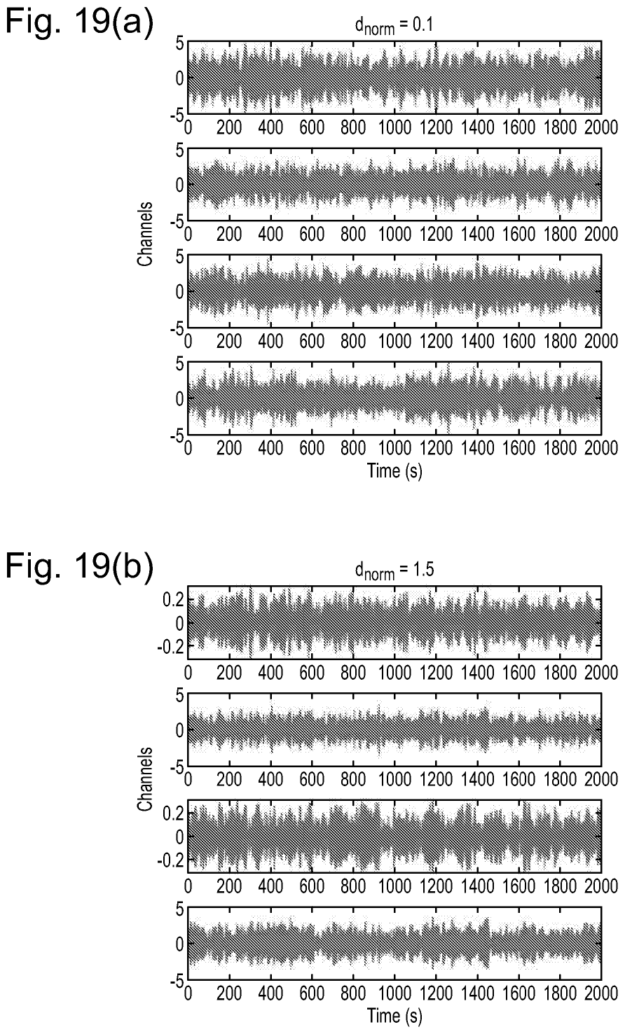

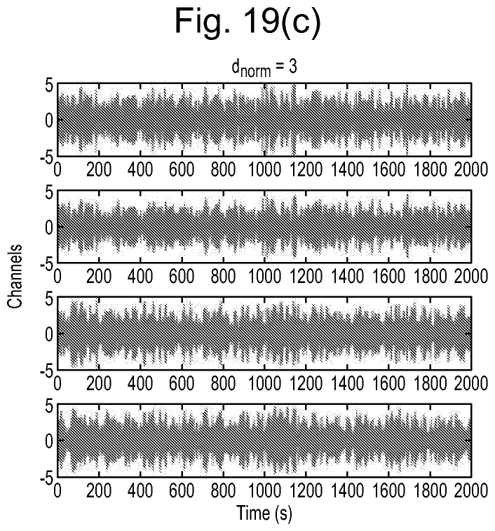

FIG. 19 shows simulated sensor recordings due to the activities of neural populations;

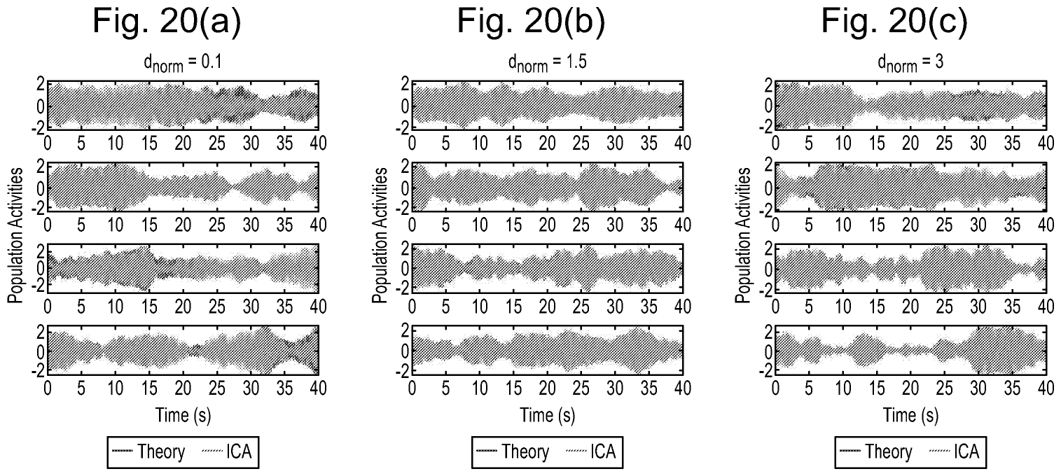

FIG. 20 shows estimates of the activities of the neural populations derived from ICA applied to the signals of FIG. 19; and

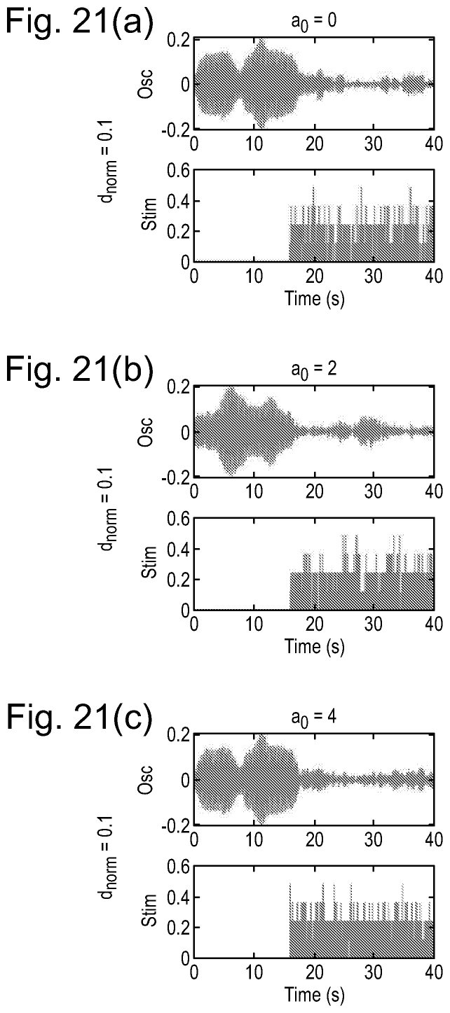

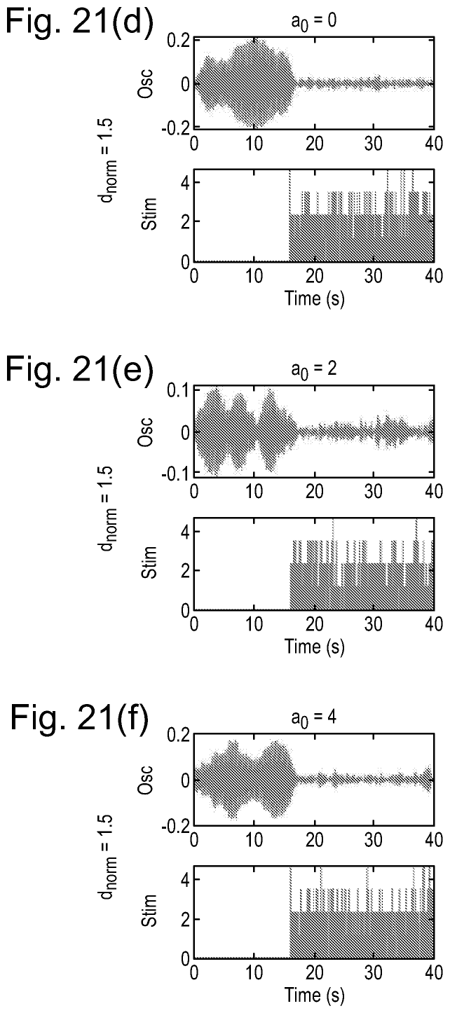

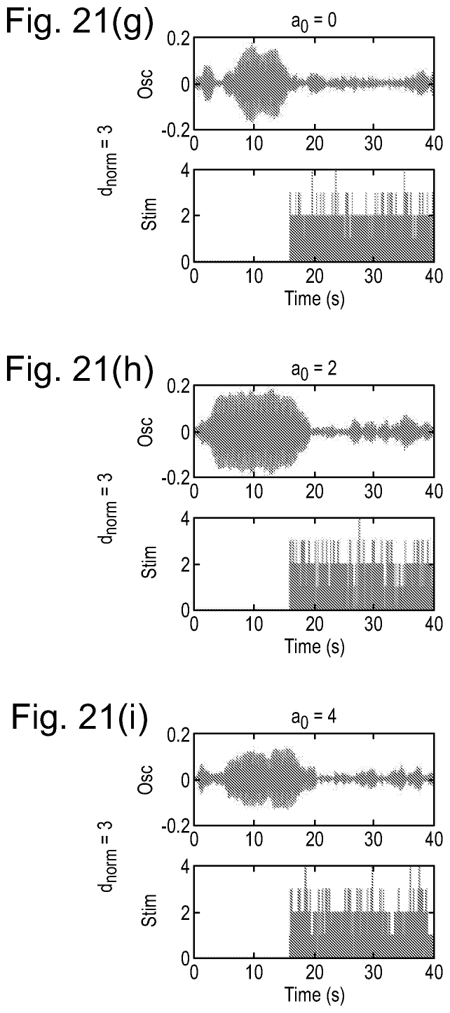

FIG. 21 shows simulated symptom signals for several configurations after applying stimulation signals.

### 1. DESCRIPTION OF THE INVENTION

The present invention provides methods of generating stimulation signals, and a system 1 for applying stimulation signals as shown in FIG. 1. The stimulation method described herein is referred to as adaptive coordinated reset (ACR) stimulation, or alternatively as adaptive coordinated desynchronization (ACD). As discussed above, stimulation signals are an effective treatment for neurological conditions including PD and ET. Therefore, in some embodiments, the application of the stimulation signals is used for treatment of Parkinson's disease and/or essential tremor. The stimulation signals may also be used treatment of other conditions that are known to be treatable using deep brain stimulation, for example epilepsy or obsessive compulsive disorder.

The system 1 comprises a processor 3 configured to generate a plurality of stimulation signals according to the method, which will be discussed in further detail below. The system 1 further comprises an electrical circuit 5 configured to provide the plurality of stimulation signals to a corresponding plurality of electrodes 7 for application at the plurality of target sites. In some embodiments, the plurality of target sites are in the brain 15 of a subject 13, and the stimulation signals comprise deep brain stimulation signals. In such embodiments, the plurality of electrodes 7 are configured to be implanted into the brain 15 of the subject 13, as shown in FIG. 2. In other embodiments, the stimulation signals may be applied to the peripheral nervous system, and the electrodes implanted in a subject's limbs, or the electrodes may be attached externally to the subject. In practice, the stimulation can be applied either using multiple electrodes 7, or single electrodes with multiple contacts. Therefore the terms ‘electrode’ and ‘contact’ are synonymous in the context of this application.

The processor 3 and electrical circuit 5 are contained in a casing 11. The casing 11 may be worn external to the body of the subject 13, or in some embodiments may be implanted into the body of the subject 13. The electrical circuit 5 in FIG. 1 comprises the plurality of electrodes 7. This is advantageous because the properties of the electrodes can be chosen to suit the properties of the system 1 and electrical circuit 5. However, this is not essential, and in some embodiments the electrodes 7 may be separate from the system 1. This may be advantageous if the subject has pre-existing electrodes already implanted, for example if they have been using a previous or alternative system for applying stimulation signals. Where the electrodes 7 are separate, the electrical circuit 5 may be configured to connect to the electrodes 7 via a suitable wired or wireless connector.

As shown in the flowchart of FIG. 15, the method of generating stimulation signals comprises receiving S10 a plurality of sensor signals from a corresponding plurality of sensors 9 on or in the subject 13. Correspondingly, the system 1 in FIG. 1 comprises a plurality of sensors 9 configured to be placed on or in the subject 13. The sensors 9 are configured to generate the plurality of sensor signals, and transmit the sensor signals to the processor 3. In some embodiments, the sensors 9 are configured to be implanted into the brain 15 of the subject 13, and the plurality of sensor signals represent electrical activity in the brain of the subject 13. The electrical activity may be a local field potential (LFP) produced by neurons in a region of the brain of the subject. Measuring a local field potential, rather than for example a potential from a single neuron or a small number of neurons, means that macroelectrodes can be used, which are larger and thereby easier to handle and implant.

As shown in FIG. 2, the plurality of sensors 9 of the system 1 are the plurality of electrodes 7. This has the advantage that it is not necessary to implant another set of components into the subject 13, and that the sensor signals produced by the sensors 9 are indicative of the electrical activity in the same regions of the brain 15 that the stimulation signals will be applied to. However, in other embodiments, the sensors 9 used to generate the sensor signals may be separate from the electrodes 7 used to apply the stimulation signals.

Electrodes used to generate sensor signals are susceptible to recording the stimulation pulses themselves. This manifests in recordings as an artefact, which poses a challenge for closed-loop methods that rely on the real-time measurement of phases and amplitudes. Therefore, in some embodiments, suppression of stimulation artefacts may be applied to the sensor signals. This suppression may come as a by-product of using independent component analysis on the signals (which is discussed further below), as previously seen [23, 24]. Alternatively, by recording through two contacts adjacent to a single stimulating contact, the properties of differential amplifiers can be used to suppress the stimulation artefact [25].

In some embodiments, the sensors 9 are inertial sensors, and the plurality of sensor signals are inertial signals representing movement of the subject 13. This can be advantageous because the movement of the subject 13 can provide a signal that is a more direct measure of symptom severity.

The method comprises using the received sensor signals to generate a plurality of stimulation signals. This type of feedback-based control of the stimulation signals is known as closed-loop control. Closed-loop DBS strategies are characterised by their use of a feedback signal to determine when stimulation should be applied. The choice, use and accuracy of this feedback signal therefore plays a crucial role in determining the efficacy of a particular strategy. Both the local field potential (LFP), a measure of electrical activity in a region of the brain, and tremor (derived from inertial measurements of a subject's movement) have previously been used as feedback signals, with studies showing the effects of DBS to be dependent on both the phase and amplitude of the oscillations at the time of stimulation [7, 2]. In adaptive DBS, high frequency stimulation is applied only when the amplitude of oscillations exceeds a certain threshold [2] and in phase-locked DBS stimulation is applied according to the instantaneous phase of the oscillations, which for ET patients corresponds to stimulation at roughly the tremor frequency (typically ˜5 Hz) [7]. Although closed-loop strategies for DBS have been used previously, none have attempted to generate a plurality of stimulation signals for simultaneous application to a plurality of target sites. In contrast, the present method (ACR) is a method for DBS using multiple contacts. Using a plurality of stimulation signals has the advantage of allowing for greater control of the field applied to the brain and thereby providing more effective treatment. Unlike known coordinated reset (CR) stimulation techniques, the ACR method is closed-loop and uses information about the neuronal system to determine when to apply stimulation. The numerical simulations presented below show that in many cases, substantial improvements to the efficacy can be achieved with ACR over existing methods.

Typically, the stimulation signals comprise a plurality of pulses. These are usually electrical pulses, but this is not essential, and in some embodiments, the pulses may be magnetic or may be provided using optogenetic techniques, where light pulses are used to perturb genetically modified neurons. An optogenetic approach to stimulation may be preferable in some embodiments, as it would eliminate the electrical stimulation artefacts mentioned above. In general terms, any form of stimulation may be used which can perturb the firing rate of neurons.

The stimulation signals are configured to deliver energy to the target sites, and the rate of energy delivery can be varied by changing the properties of the applied signal. For example, the amplitude, and frequency of the signal can be used to vary the rate of energy delivery to the target sites. In some embodiments, the stimulation signals have a carrier frequency of at least 20 Hz, preferably at least 50 Hz, more preferably at least 100 Hz. In some embodiments, the stimulation signals have a carrier frequency of at most 250 Hz, preferably at most 200 Hz, preferably at most 150 Hz.

The generating of the plurality of stimulation signals comprises using a model of the response of neurons in the subject 13 to the stimulation signals. Using such a model allows for more accurate determination of symptom severity and neural activity, and thereby the tailoring of the stimulation signals to more effectively treat the symptoms. This is particularly advantageous when using closed-loop control to generate a plurality of stimulation signals for simultaneous application to a plurality of target sites, where the problem of determining optimal stimulation signals is particularly complex.

In [8], a mathematical basis for the phase and amplitude dependence of single-contact DBS was derived. This is discussed briefly below to provide context for the model of the present invention. The present model extends the model discussed in [8] to provide a more detailed model that also covers multi-electrode stimulation, and the present model is then applied to the practical problem of generating stimulation signals to introduce adaptive coordinated reset (ACR), a closed-loop stimulation strategy especially suited to multi-contact systems. A key aspect of the present model is that it models neural tissue, for example brain tissue or tissue of the peripheral nervous system, as a plurality of coupled populations of neurons. This allows the understanding of how the effects of multi-contact stimulation (and in particular DBS) depend on the ongoing neural activity measured by the plurality of sensor signals through each channel from each sensor.

### 2. MODEL FOR SINGLE CONTACT DBS

### 2.1 Phase Synchrony and Oscillations

This section follows the derivation in [8] and considers a model of how stimulation with a single electrode acts on a population of neurons. A list of frequently used notation is provided in Table 1.

TABLE 1
List of frequently used symbols together with their description.

| Parameter | Description |
|---|---|
| k | Coupling constant |
| \tilde{σ} | Noise amplitude |
| ω̅ | Mean of natural frequencies |
| s_{ω} | Standard deviation of natural frequencies |
| Z | Neuronal phase response curve (nPRC) |
| a | Even Fourier coefficient of nPRC |
| b | Odd Fourier coefficient of nPRC |
| θ | Phase of oscillator |
| ψ | Phase of population |
| ρ | Synchrony of population |
| r | Complex order parameter |
| c | Scaling constant for experimental data |
| S | Number of populations |
| L | Number of electrodes |
| N | Number of neurons |
| J | Number of constraints |
| Γ | Amplitude response for a single population |
| w | Population weight |
| dₙₒᵣₘ | Electrode-population distance |
| p′ | Electrode position |
| p | Neuron position |
| P | Population position |
| v′ | Voltage at electrode |
| v | Voltage at neuron |
| V | Voltage at population |
| q′ | Charge of electrode |
| q | Charge of neuron |
| Q | Charge of population |
| D | Activity to voltage at electrode transformation matrix |
| \tilde{D} | Electrode to voltage at population transformation matrix |
| d | Element of D |
| \tilde{d} | Element of \tilde{D} |

The amplitude measured in feedback signals (i.e. the sensor signals from the plurality of sensors) can be related to the synchrony of neural populations. The instantaneous phase Ψ(t) and envelope amplitude P(t) of a signal F(t) can be obtained using the analytic signal R(t)

R(t)=Pe^{iψ}=F(t)+iĤ[F(t)], (1)

where Ĥ denotes the Hilbert transform. This quantity can be related to those associated with a state of oscillators. The state of N regular spiking neurons is defined to be given by a set of N oscillators each with phase θₙ(t), which are the phases describing where each neuron is in its firing cycle. The phase synchrony of this system can be measured using the order parameter r, defined to be

$$r = ρ {e}^{i ψ} = \frac{1}{N} {\sum }_{n = 1}^{N} {e}^{i {θ}_{n}} \qquad ( 2 )$$

The above definition ensures the magnitude of the order parameter ρ can take values between 0 and 1, representing full desynchrony and full synchrony, respectively. We can transform the state of the system to a signal representing the neural activity using a superposition of cosine functions

$$f ( t ) = Re ( r ) = \frac{1}{N} {\sum }_{n = 1}^{N} {e}^{i {θ}_{n}} . \qquad ( 3 )$$

The choice of a cosine function is for mathematical convenience since it corresponds to the real part of (2). In addition to this, the cosine function has a maximum at 0, and in classic coupled oscillator models, phase 0 corresponds to the phase when neurons produce spikes [9]. Hence post-synaptic potentials in down-stream neurons receiving an input from the modelled population will be a smoothed function of spikes produced in phase 0, so the cosine function captures key features of such post-synaptic potentials. Using the Euler relation and comparing (3) with the real part of (2) shows

f(t)=ρcos(ψ) (4)

A simple relationship is assumed between the neural activity f(t) and a measured feedback signal, for example tremor and the LFP

F(t)=cf(t) (5)

where the measured signal is denoted by F(t). This is reasonable in the case of ET, where thalamic activity is known to be highly correlated to tremor [3]. Inserting Eq. (5) into (1) gives

Pe^{iψ}=c{f(t)+iĤ[f(t)]}. (6)

Inserting Eq. (3) into Eq. (6) and using the linearity of Ĥ leads to

$${Pe}^{i Φ} = \frac{c}{N} {\sum }_{n = 1}^{N} \{ cos ( {θ}_{n} ) + i \hat{H} [ cos ( {θ}_{n} ) ] \} . \qquad ( 7 )$$

Under the reasonable assumption that the time evolution of θₙ is monotonic, it can be shown [8] that

Ĥ[cos(θₙ)]≅sin(θₙ), (8)

and therefore

$${Pe}^{i Φ} = \frac{c}{N} {\sum }_{n = 1}^{N} \{ cos ( {θ}_{n} ) + i sin ( {θ}_{n} ) \} = c {ρe}^{i Φ} . \qquad ( 9 )$$

hence, the instantaneous envelope amplitude and phase (the analytic signal) is relatable to the magnitude and phase of the order parameter using

P=cρ, Ψ=ψ (10)

In summary, assuming the experimental data and neural activity are related according to Eq. (5) and that the phases {θn} increase monotonically with time, the Hilbert transform of the experimental data can be used to relate the envelope amplitude and instantaneous phase to the magnitude and phase of the order parameter, respectively.

### 2.2. Response Curves

The neuronal phase response curve (nPRC) for a spiking neuron is the change in spike timing due to a perturbation as a function of the inter-spike time. Hansel et al [10] categorised nPRCs into either type I or type II depending on whether a small excitatory (inhibitory) input always advances (delays) a neuron to a next spike or whether it either advances or delays a spike, depending on where the neuron is in its firing cycle, respectively [11]. These effects of inputs can be captured using a simple mathematical function Z(θ). By mapping where a neuron is in its firing cycle onto a phase variable θ∈[0,2π], the nPRC describes the change in phase of a single neuron due to a stimulus. More precisely, under the assumption of a weak input ϵU(t), the evolution of a single oscillator can be written in terms of a natural frequency ω₀ in addition to a response term

$$\frac{d θ}{dt} = {ω}_{0} + ε U ( t ) Z ( θ ) . \qquad ( 11 )$$

A general neuronal nPRC can be expanded as a Fourier series

$$Z ( θ ) = \frac{{a}_{0}}{2} + {\sum }_{m = 1}^{∞} {a}_{m} cos ( m θ ) + {\sum }_{m = 1}^{∞} {b}_{m} sin ( m θ ) . \qquad ( 12 )$$

The nPRC type is reflected in the zeroth harmonic α₀, or the shift, with |α0| large and small relative to the other coefficients being indicative of type I and type II curves, respectively. Phase oscillator models which incorporate the nPRC can be shown to reproduce the experimentally-known characteristics of a patient's response to stimulation [8], namely that the effects should be both amplitude and phase dependent [2, 7]. This leads to the concept of the phase response curve (PRC) and the amplitude response curve (ARC) for feedback signals, such as LFP and tremor, which can be described by perturbing a population of oscillators and respectively describe changes in the phase and amplitude of the feedback signal at the point of stimulation. The instantaneous curves, which are functions of both the phase and amplitude at which the stimulation is delivered, are not commonly found in experimental analysis due primarily to the difficulties associated with obtaining a function of two independent variables from noisy data. It is more common to find the averaged response curves, which are only functions of the phase and are averaged over the amplitude. Such curves are readily obtainable using standard signal processing techniques and have been used to characterise a patient's response to stimulation [12, 7, 13].

### 2.3. The Kuramoto Model

Modelling the effects of DBS generally poses a challenge since the brain networks involved in disorders such as ET (cortico-thalamic circuit) and PD (cortico-basal-ganglia circuit) are complex and it is still debated from which parts of these circuits the pathological oscillations originate. The task can be made more tractable by considering a phenomenological model which does not attempt to explicitly describe the underlying circuits, but rather focuses on general mechanisms leading to the synchronization of neurons. To achieve this, the model may model each population of neurons as a plurality of coupled oscillators.

One example of this is the Kuramoto model, where the dynamics of neurons are described using a system of homogeneously coupled oscillators, whose phases evolve according to a set of underlying differential equations. Such models are particularly attractive due to their simplicity and explicit dependence on phase, which makes them convenient for describing the effects of phase-locked stimulation. In the previous section it was shown that the oscillation data typically measured in experiment can be modelled using an underlying system of oscillators, whose state is described by the set of N phases. The time evolution of this state (for a single population) can be described using the Kuramoto equations, with an additional term describing the effects of stimulation [5]

$$\frac{d {θ}_{n}}{dt} = {ω}_{n} + \frac{k}{N} {\sum }_{m = 1}^{N} sin ( {θ}_{m} - {θ}_{n} ) + V ( t ) Z ( {θ}_{n} ) . \qquad ( 13 )$$

The first term of (13) is the natural frequency ωₙ which describes the frequency in the absence of external inputs. The second term describes the coupling between the activity of individual neurons, where k is the coupling constant which controls the strength of coupling between each pair of oscillators and hence their tendency to synchronize. The third term describes the effect of stimulation, where the intensity of stimulation is denoted by V(t). The nPRC denoted by Z(θₙ), describes a neuron's sensitivity to stimulation at a particular phase and reflects the observation that the effects of stimulation depend on where a neuron is in its firing cycle [14]. By using the nPRC, the model models the response of the neurons to the stimulation signals as being dependent on the phase of oscillations of the neurons. Using the definition of the order parameter Eq. (13) can be transformed to give

$$\frac{d {θ}_{n}}{dt} = {ω}_{n} + k ρ sin ( φ - {θ}_{n} ) + V ( t ) Z ( {θ}_{n} ) . \qquad ( 14 )$$

In this form, it is clear that each oscillator has a tendency to move towards the population phase W and that the strength of this tendency is controlled by the coupling parameter k.

### 2.4. Reduced Kuramoto Model

In the previous section, the dynamics of a finite system of oscillators was described using the Kuramoto equations. In this model, stimulation is described as a perturbation to the phase of an oscillator, with each oscillator experiencing a different effect of stimulation depending on its phase (and determined by Z(θ)). Stimulation therefore has the effect of changing the distribution of oscillators and hence the order parameter of the system. Since the order parameter is determined by both the amplitude and phase of the system, the expectation is that stimulation will lead to a change in both these quantities, which is referred to as the instantaneous amplitude and phase response of the system. To obtain analytical expressions for these quantities, an infinite system of oscillators is considered satisfying the ansatz of Ott and Antonsen [16, 17]. In [8], it was shown that for a general nPRC given by Equation (12) and assuming the natural frequencies are Lorentzian distributed with centre ω₀ and width γ, the instantaneous change in the order parameter can be written as

$$\frac{dr}{dt} = ( i {ω}_{0} - γ ) r + \frac{kr}{2} ( 1 - {\vert r \vert}^{2} ) + \frac{iV ( t )}{2} \{ {a}_{0} r + {\sum }_{m = 1}^{∞} {a}_{m} [ {( {r}^{*} )}^{m - 1} + {r}^{m + 1} ] + i {\sum }_{m = 1}^{∞} {b}_{m} [ {( {r}^{*} )}^{m - 1} - {r}^{m + 1} ] \} . \qquad ( 15 )$$

$$Using this , expressions for the ARC and PRC due to stimulation are$$

$$\frac{d {ρ}_{stim}}{dt} = \frac{V ( t )}{2} ( 1 - {ρ}^{2} ) {\sum }_{m = 1}^{∞} {ρ}^{m - 1} [ {a}_{m} sin ( m φ ) - {b}_{m} cos ( m φ ) ] , \qquad ( 16 )$$

$$and$$

$$\frac{d {φ}_{stim}}{dt} = \frac{V ( t )}{2} \{ {a}_{0} + ( 1 + {ρ}^{- 2} ) {\sum }_{m = 1}^{∞} {ρ}^{m} [ {a}_{m} cos ( m φ ) + {b}_{m} sin ( m φ ) ] \} . \qquad ( 17 )$$

### 3. REPRODUCING TREMOR IN ET PATIENTS

To demonstrate that the Kuramoto model can produce oscillations which are compatible with tremor data from ET patients, the Kuramoto model is fitted to tremor data [7, 19] from ET patients deemed to have significant response curves [18]. To account for random forces which may influence the firing of individual neurons, the Kuramoto model can be extended to include a noise term, which is taken to be a Wiener process. The time evolution for θₙ then becomes

dθₙ=[ωₙ+kρ sin(ψ−θₙ)+V(t)Z(θₙ)]dt+\tilde{σ}N(0,1)\sqrt{dt}, (18)

where \tilde{σ} is the noise amplitude and N(0,1) is a random number sampled from a standard normal distribution. The parameters found through optimisation are provided in Table 2. The parameters were found by fitting the model to tremor data taken from ET patients by Cagnan et al [7].

TABLE 2
Parameters for the single population Kuramoto model given by Equation (18).

| Patient | σ̅ | k | ω̅/2π | s_{w}/2π | V | α₀ | α₁ | b₁ |
|---|---|---|---|---|---|---|---|---|
| 1 | 2.78 | 9.15 | 4.96 | 0.31 | 0.06 | −0.01 | −0.01 | −0.05 |
| 5 | 3.10 | 14.11 | 4.24 | 0.68 | 0.22 | −0.12 | 0.01 | −0.02 |
| 6 | 1.58 | 1.57 | 3.88 | 0.44 | 0.15 | −0.07 | −0.02 | 0.01 |

FIG. 3 shows fits to various features extracted from the oscillation data. The first row (a)-(c) is the power spectral density (PSD), the second row (d)-(f) is the probability density function (PDF) for the envelope amplitude and the third row (g)-(i) is the PSD of the envelope. Columns (a)-(g), (b)-(h) and (c)-(i) are for patients 1, 5 and 6, respectively. FIG. 4 shows a comparison between the averaged response curves for experimental data and the fitted Kuramoto model. The phase response curve was used as a feature during the fitting procedure. The amplitude response curve is predicted from the model. Columns (a)-(d), (b)-(e) and (c)-(f) are for patients 1, 5 and 6, respectively. FIGS. 3 and 4(a)-(c) show the Kuramoto model is able to fit well to the features taken from the experimental data. FIGS. 4(d)-(f) show that the fitted model is able to reproduce the amplitude response for patients generally well, although FIG. 4(d) does show a noticeable phase shift between the simulated and experimental curves for Patient 1.

FIG. 5 shows a comparison between experimentally measured tremor data [7] and output from the fitted Kuramoto model. Columns (a)-(d), (b)-(e) and (c)-(f) are for patients 1, 5 and 6, respectively. The output from the model in FIG. 5 shows the resulting simulated data to be quite compatible with that found from experiment. The model can be seen to capture the basic properties of the experimental data, but not the more exotic features, such as the sustained periods of lower amplitudes, which are likely due to non-stationarity. Overall, our findings suggest that it is reasonable to use the Kuramoto model as a model for tremor in ET patients. It is on this basis that we derive the expressions for the response curves in subsequent sections.

### 4. MULTI-CONTACT DBS

### 4.1. Multi-Population Kuramoto Model

This section shows that modelling a symptom due to excessive synchrony of multiple neural populations can be achieved by extending the concepts presented in sections 2.1 and 2.3. To achieve this, the model models neural tissue as a plurality of coupled populations of neurons. The set of oscillators representing the neurons can be arbitrarily divided into S populations with N_{σ} oscillators for the σth population. The order parameter defined by Equation (2) can then be rewritten using a double summation

$$r = \frac{1}{N} {\sum }_{σ = i}^{S} {\sum }_{n = 1}^{{N}_{σ}} {e}^{i θσ n} , \qquad ( 19 )$$

with oscillator n of population a being denoted by θ_{σn}. The factor of

$$\frac{1}{N}$$

can be brought inside the first summation and rewritten as

$$\frac{N σ}{N σ N} .$$

Then, with

$${w}_{σ} = \frac{N σ}{N} \qquad ( 20 )$$

the order parameter for the system can be written as

$$r = {\sum }_{σ = 1}^{S} \frac{{w}_{σ}}{{N}_{σ}} {\sum }_{n = 1}^{{N}_{σ}} {e}^{i {θ}_{σ n}} . \qquad ( 21 )$$

Using the definition of the order parameter (2), Eq. (21) can be written as a weighted superposition of the order parameters for each population

$$r = {\sum }_{σ = 1}^{S} {w}_{σ} {r}_{σ} , , \qquad ( 22 )$$

$$with$$

$${\sum }_{σ = 1}^{S} {w}_{σ} = 1. \qquad ( 23 )$$

The global order parameter is defined as r with amplitude ρ and phase ψ and the local order parameter defined as r_{σ} for population σ with amplitude ρ_{σ} and phase ψ_{σ}. The importance of the global order parameter is that its magnitude ρ is a measure of total synchrony and hence should be highly correlated to the severity of a symptom, such as tremor in the case of ET. In the case of PD, symptom severity could be measured using the unified Parkinson's disease rating scale (UPDRS) scores [20]. Therefore, stimulation should be defined to maximally reduce the magnitude of the global order parameter.

Eq. (22) can also be related to feedback signals measured by using (3) and taking the real part. Under the assumption (5) relating the neural activity to the feedback signal, the feedback signal in terms of population activities is

$$F ( t ) = {\sum }_{σ = 1}^{S} {cw}_{σ} {f}_{σ} ( t ) . \qquad ( 24 )$$

where F(t) and f_{σ}(t) are the global signal and local signals (or population activities), respectively. Therefore in this part of the method, generating the plurality of stimulation signals comprises determining S14 a population activity for each population of neurons, and determining the amplitude and phase of each population activity. The population activity of each population is a measure of neural activity among neurons in that population. Using (4), Equation (24) can also be written in terms of the global and local amplitudes and phases

$$P cos ( φ ) = {\sum }_{σ = 1}^{S} {cw}_{σ} {ρ}_{σ} cos ( {φ}_{σ} ) . \qquad ( 25 )$$

The global signal Re(F(t))=Pcos(ψ) may also be referred to as a composite signal, and therefore generating the plurality of stimulation signals comprises determining S16 a composite signal using a weighted combination of the amplitudes and phases of the population activities, and determining the amplitude and phase of the composite signal. The Kuramoto equations (13) can also be rewritten in terms of the population phases ψ_{σ} and amplitudes ρ_{σ}

$$\frac{d {θ}_{σ n}}{dt} = {w}_{σ n} + {\sum }_{{σ}^{′} = 1}^{S} {w}_{{σ}^{′}} {k}_{{σσ}^{′}} {ρ}_{{σ}^{′}} sin ( {φ}_{{σ}^{′}} - {θ}_{σ n} ) + {V}_{σ} ( t ) {Z}_{σ} ( {θ}_{σ n} ) , \qquad ( 26 )$$

where V_{σ}(t) is now the stimulation intensity at a population σ. The coupling constant k in Eq. (13) is now a S×S matrix with elements k_{σσ′}. The diagonal and off-diagonal elements describe the intrapopulation and interpopulation coupling, respectively.

### 4.2. Multi-Population Response Curves

The change in the global amplitude due to stimulation can be expressed as a function of the local (population) amplitudes and phases. It is assumed that the local quantities (to base the stimulation on) can be measured. How these quantities are measured will be discussed in further detail later.

Using the polar form of the order parameter (2), Equation (22) can be written as a summation involving the amplitudes and phases of individual populations.

$$ρ {e}^{i φ} = {\sum }_{σ = 1}^{S} {w}_{σ} {ρ}_{σ} {e}^{i {φ}_{σ}} , \qquad ( 27 )$$

Taking the time derivative of (27) leads to

$$\frac{d ρ}{dt} + i ρ \frac{d φ}{dt} = {\sum }_{σ = 1}^{S} {w}_{σ} [ \frac{d {ρ}_{σ}}{dt} + i {ρ}_{σ} + \frac{d {φ}_{σ}}{dt} ] {e}^{i ( {φ}_{σ} - φ )} , \qquad ( 28 )$$

which can be written in terms of the real and imaginary components as

$$\frac{d ρ}{dt} + i ρ \frac{d φ}{dt} = {\sum }_{σ = 1}^{S} {w}_{σ} \{ [ \frac{d {ρ}_{σ}}{dt} cos ( {φ}_{σ} - φ ) - {ρ}_{σ} \frac{d {φ}_{σ}}{dt} sin ( {φ}_{σ} - φ ) ] + i [ {ρ}_{σ} \frac{d {φ}_{σ}}{dt} cos ( {φ}_{σ} - φ ) + \frac{d {ρ}_{σ}}{dt} sin ( {φ}_{σ} - φ ) ] \} . \qquad ( 29 )$$

The real part is the time derivative of the amplitude of the global order parameter

$$\frac{d ρ}{dt} = {\sum }_{σ = 1}^{S} {w}_{σ} [ \frac{d {ρ}_{σ}}{dt} cos ( {ψ}_{σ} - ψ ) - {ρ}_{σ} \frac{d {ψ}_{σ}}{dt} sin ( {ψ}_{σ} - ψ ) ] . \qquad ( 3 )$$

The quantities

$$\frac{d {ρ}_{σ}}{dt} and \frac{d {ψ}_{σ}}{dt}$$

and are the changes in the amplitude and phase of a population with respect to time. The distribution phases within a population is assumed to satisfy the ansatz of Ott and Antonsen [16], and thereby the amplitude response due to stimulation in terms of the Fourier coefficients of Z(θ) is

$$\frac{d {ρ}_{stim}}{dt} = \frac{1}{2} {\sum }_{σ = 1}^{S} {w}_{σ} {V}_{σ} ( t ) \{ {\sum }_{m = 1}^{∞} {ρ}_{σ}^{m - 1} [ {a}_{m} sin [ ( m - 1 ) {ψ}_{σ} - ψ ] - {b}_{m} cos [ ( m - 1 ) {ψ}_{σ} + ψ ] ] - {\sum }_{m = 0}^{∞} {ρ}_{σ}^{m + 1} [ {a}_{m} sin [ ( m + 1 ) {ψ}_{σ} - ψ ] - {b}_{m} cos [ ( m + 1 ) {ψ}_{σ} - ψ ] ] \} , \qquad ( 31 )$$

where, for simplicity, it is assumed that Z(θ) is the same for all populations. Equation (31) contains an expansion over the harmonics of Z(θ). In [8] it was demonstrated that, for a biologically realistic nPRC, it is reasonable to neglect higher harmonic terms (m>1), leading to a simpler expression for the instantaneous amplitude response

$$\frac{d {ρ}_{stim}}{dt} ≃ \frac{1}{2} {\sum }_{σ = 1}^{S} {w}_{σ} {V}_{σ} ( t ) \{ [ {a}_{1} sin ( ψ ) - {b}_{1} cos ( ψ ) ] - {ρ}_{σ} {a}_{0} sin ( {ψ}_{σ} - ψ ) - {ρ}_{σ}^{2} [ {a}_{1} sin ( 2 {ψ}_{σ} - ψ ) - {b}_{1} cos ( 2 {ψ}_{σ} - ψ ) ] \} , \qquad ( 32 )$$

Equation (32) shows the global reduction in amplitude can be expressed as a sum of contributions from each population, with each term dependent on 3 variables: the global phase ψ, the local phase ψ_{σ} and the local amplitude ρ_{σ}. It also suggests that stimulating on the basis of local quantities may not always be advantageous. It can be seen that the terms of Equation (32) can be divided into two categories: ones which depends on both global and local quantities and ones which depends only on global quantities. The terms depending on both the global and local phases are also dependent on the local amplitudes. In cases where the local amplitude is small, i.e. ρ_{σ}<<1, the term involving ρ_{σ}² can be neglected, leading to a simplified expression

$$\frac{d {ρ}_{stim}}{dt} ≃ \frac{1}{2} {\sum }_{σ = 1}^{S} {w}_{σ} {V}_{σ} ( t ) \{ [ {a}_{1} sin ( ψ ) - {b}_{1} cos ( ψ ) ] - {ρ}_{σ} {a}_{0} sin ( {ψ}_{σ} - ψ ) \} . \qquad ( 33 )$$

Here, it can be seen that the amplitude response depends only on the global phase if the zeroth harmonic of the nPRC α₀ is negligible, which is the case for type II nPRCs. It can also be seen that the dependency of the amplitude response on the local quantities of population a becomes less at increasingly lower local amplitudes ρ_{σ}. In addition to this, the dependence on sin(ψ_{σ}−ψ) implies that stimulating on the basis of local quantities would only have an effect if the phases of individual populations differ sufficiently from the mean phase. One situation in which such phase difference may be particularly high are for clustered configurations of oscillators. Examples of different configurations of oscillators are shown in FIG. 6, colour coded according to population. FIG. 6 shows (a) unimodal distribution, and (b) multimodal (clustered) distribution. Configurations were obtained by simulating the multi-population Kuramoto equations (26).

FIG. 7 shows the predicted contribution of a single population to the amplitude response at different local amplitudes ρ_{σ} according to Eq. (32). Each panel corresponds to a single ET patient from the study of Cagnan et al [7], where the Fourier coefficients of the nPRC were determined using a fitting procedure. Panels (a) (b) and (c) are for patients 1, 5 and 6, respectively. For each plot, the vertical axis is the global phase (ψ) and the horizontal axis is the local (or population) phase (ψ_{σ}). The corresponding nPRC Z(θ) is also shown, with zero indicated by a red dashed line. Blue regions indicate areas where stimulation is predicted to suppress amplitude. For a given local amplitude, a single term is plotted from the summation over populations in Equation (32). This provides the contribution of a single population to the amplitude response as a function of the local and global phases.

Regions in blue are areas of amplitude suppression while orange regions predict amplification. In both cases, these regions can be seen to occur in bands. Graphically, the dependence of the amplitude response on the global and local phases can be inferred from the direction of the banding. A purely horizontal band implies the amplitude response is independent of the local phase. An example of this can be seen at low amplitudes in FIG. 7(a). Other plots show diagonal banding, which implies the amplitude response is dependent on both the global and local phases. This behaviour can be understood by considering the 3 terms of (32). At low amplitudes, the first term dominates, which is only dependent of the global phase. As the local amplitude increases, the second and third terms depending on local quantities become increasingly more important. For the cases where |α₀| is small, the effect is less apparent. The left panel of FIG. 7 (a) shows that stimulation can either increase or reduce the phase (i.e. an nPRC of type II), implying a relatively small |α₀|. Hence, for this patient, the second and third terms are negligible, except at higher amplitudes. FIGS. 7 (b) and (c) shows that stimulation has the effect of only increasing the phase, which is indicative of Z(θ) with larger |α₀|. For these systems the amplitude response can be seen to depend more strongly on the local phase for all amplitudes.

### 4.3. Obtaining Population Activities Through Electrode Measurements

The local phases {ψ_{σ}} and amplitudes {ρ_{σ}} can be recovered using LFP measurements through different contacts. This requires incorporating information about the geometry of the electrode placement into the equations for the response curve in addition to assigning a physical interpretation to the population activity. The aim is not to construct a detailed electrophysiological model of neuronal activity, but rather to present a general form for the voltage measured at an electrode contact.

The expressions are formulated in terms of electric charge, but the same form also permits the use of currents. The mathematical form of the equations is unchanged whether they are formulated in terms of charge (q) or current (I). In addition to this, the expressions include summations over neurons, but an equally valid expression can be made by summing over elements of space, as is the case in multi-compartmental models [21]. The quantities in the model are voltages ν′ₗ(t) measured at electrode l due to the activity of population a producing charges Q_{σ}(t) and voltages V_{σ}(t) at population σ due to stimulation which delivers charge q′ₗ(t) to electrode l. The voltage V_{σ}(t) can also be thought of as the ‘stimulation intensity’ experienced at population σ.

First, consider a system of L electrodes and N neurons with positions in space denoted by p′ and p, respectively. Primed symbols denote quantities associated with electrodes, lower case symbols represent neuronal quantities, and upper case symbols represent population quantities.

Voltages measured at an electrode arise due to the geometry of the electrode-neuron system and the intrinsic electrical activity of each neuron. The voltage measured at an electrode is expressed in terms of a summation over charges due to the neurons qₙ(t)

$${v}_{l}^{′} ( t ) = {\sum }_{n = 1}^{N} d ( {p}_{l}^{′} , {p}_{n} ) {q}_{n} ( t ) , \qquad ( 34 )$$

where d(p′ₗ, pₙ) are coefficients which reflect the medium and geometry of the electrode-neuron system. For example, in the case of a coulombic system, the coefficients would be

$$d ( {p}_{l}^{′} , {p}_{n} ) = \frac{{κ}_{e}}{\vert {p}_{l}^{′} - {p}_{n} \vert} \qquad ( 35 )$$

where κₑ is the Coulomb constant. As above, the neurons can be arbitrarily divided into S populations, with each neuron referenced by both a population and position index σ and n, respectively.

$${v}_{l}^{′} ( t ) = {\sum }_{σ = 1}^{S} {\sum }_{n = 1}^{{N}_{σ}} d ( {p}_{l}^{′} , {p}_{σ n} ) {q}_{σ n} ( t ) , \qquad ( 36 )$$

The position of a neuron is redefined as p_{σn}=P_{σ}+Δp_{σn}, i.e. in terms of a vector to a region (or population) plus a shift. Then

$${v}_{l}^{′} ( t ) = {\sum }_{σ = 1}^{S} {\sum }_{n = 1}^{{N}_{σ}} d ( {p}_{l}^{′} , {P}_{σ} + Δ {p}_{σ n} ) {q}_{σ n} ( t ) , \qquad ( 37 )$$

If the region at P_{σ} is assumed to be small, then

Δp_{σn}≈0 (38)

The potential at the electrode can then be written in terms of population activity

$${v}_{′}^{l} ( t ) = {\sum }_{σ = 1}^{S} d ( {p}_{l}^{′} , {P}_{σ} ) {Q}_{σ} ( t ) , \qquad ( 39 )$$

where

$${Q}_{σ} ( t ) = {\sum }_{n = 1}^{{N}_{σ}} {q}_{σ n} ( t ) . \qquad ( 40 )$$

The time dependent charge of a population Q_{σ}(t) can be related to the neural activity by assuming a form for q_{σn}(t), specifically that

$${q}_{σ n} ( t ) = \frac{c}{N} {\sum }_{n = 1}^{{N}_{σ}} cos ( {θ}_{σ n} ) . \qquad ( 41 )$$

Using (40) and (20) gives

$${Q}_{σ} ( t ) = \frac{{cw}_{σ}}{{N}_{σ}} {\sum }_{n = 1}^{{N}_{σ}} cos ( {θ}_{σ n} ) . \qquad ( 40 )$$

Using (3) and (4) then gives an expression for the time dependent charge of a population in terms of the population phase and amplitude

Q_{σ}(t)=cw_{σ}ρ_{σ} cos(ψ_{σ}). (43)

Using (39) the potential at the electrodes can therefore be written in matrix form

$$( \begin{matrix}{d}_{11} & {d}_{12} & {d}_{13} & … & {d}_{1 S} \\ ⋮ & ⋱ & & & \\ ⋮ & & ⋱ & & \\ ⋮ & & & ⋱ & \\ {d}_{L 1} & & & & {d}_{LS}\end{matrix} ) ( \begin{matrix}{ρ}_{1} cos ( {ψ}_{1} ) \\ {ρ}_{2} cos ( {ψ}_{2} ) \\ {ρ}_{3} cos ( {ψ}_{3} ) \\ ⋮ \\ {ρ}_{S} cos ( {ψ}_{S} )\end{matrix} ) = ( \begin{matrix}{v}_{1}^{′} ( t ) \\ {v}_{2}^{′} ( t ) \\ {v}_{3}^{′} ( t ) \\ ⋮ \\ {v}_{L}^{′} ( t )\end{matrix} ) , \qquad ( 44 )$$

where for simplicity, d_{lσ}=cω_{σ}d(p′ₗ,P_{σ}). Equation (44) can be expressed in a more compact form with D denoting the matrix of coefficients (of dimensions L×S), f as the vector of neural activities and ν′ as the vector of electrode measurements.

Df=ν′ (45)

Equation (45) relates the voltages at the electrodes ν′ to the neural activities f. In general, using Equation (32) in a closed-loop DBS strategy depends on being able to accurately measure the population quantities {ρ_{σ}} and {ψ_{σ}}. Equation (44) shows that what we actually measure at the electrodes is a linear superposition of population activities.

FIG. 8 shows visualisations of 4 electrode 4 population systems, where each population occupies a small spatial region. Each system was generated by randomly choosing the coordinates of the 4 populations so that they lie within a box of length L_{box}=10. Each electrode is then placed dₙₒᵣₘ distance from a population. Panel (a) shows a configuration where each electrode is placed very close to a population (dₙₒᵣₘ=0.5). This represents systems consisting of small separated regions of activity, with each electrode positioned close to each region. In this case, D is approximately diagonal, and the population quantities could be accurately recovered (although ρ_{σ} would be scaled). Panel (b) shows a different system (dₙₒᵣₘ=2) where both the electrodes and populations are more ‘dispersed’. In this scenario, electrodes may record activity from multiple populations.

In some embodiments, determining the population activities comprises applying S12 independent component analysis (ICA) to the sensor signals. ICA is used to resolve the S population quantities from L electrode measurements. Methods such as independent component analysis (ICA) are well-suited to solving the general problem of recovering a vector of ‘source signals’ f(t) (in this case the population activities) given a vector of recordings ν′(t), as expressed in Equation (44), although the method cannot recover the scaling.

Variations of the ICA problem exist depending on whether S<L (the overdetermined case), S>L (the underdetermined case) and S=L (the determined case). The determined case is perhaps the most common and more easily solved, since the mixing matrix D is invertible. The inverse matrix elements {g_{σl}} can then be used to easily transform the electrode measurements into estimates of the population activities {\tilde{f}_{σ}}. The estimated symptom signal \tilde{f}(t) can be calculated using a set of estimated weights {\tilde{w}_{σ}} (which would need to be found using optimisation) and the estimated population activities {\tilde{f}_{σ}}, which are found by applying the inverse matrix elements {g_{σl}} to the vector of electrode recordings ν′. The estimated symptom signal can be written as

$$\tilde{f} ( t ) = {\sum }_{σ = 1}^{S} {\tilde{w}}_{σ} {\tilde{f}}_{σ} ( t ) \qquad ( 45 a )$$

However, the value of S is not known a priori, and therefore it may not be known initially which ICA method would be best suited. If S=L is assumed, then ICA will always resolve exactly L components. With this assumption, increasing the number of electrodes in a system has a definite purpose: it increases the potential to resolve the internal state. Assuming a larger number of populations also increases the validity of the small region approximation presented in Equation (38) and thus the accuracy of ACR.

It may also be possible to obtain good approximations to the state by using L<S electrodes, since in some cases the weights w, may be small for some populations and can hence be neglected. This together with the statistical nature of ICA, errors due to applying various signal processing techniques and noise within measurement would inevitably lead to some uncertainty when determining the population quantities. In addition to this, the amplitude response (32) is also dependent on the harmonics of Z(θ), which also need to be determined.

Since in theory the matrix D should not evolve with time, ICA can be applied offline to recover D, and then used to obtain the local signals. In practice, after determining the local signals, Equation (25) can be used to construct the global signal (or composite signal). In this process, the weights {w_{σ}} should be chosen to give a global signal with an amplitude that is highly correlated to the symptom severity. Thereby, the weights in the weighted combination are such that the composite signal is correlated to a measure of severity of the symptom of the subject, preferably proportional to the measure of severity.

### 4.4. Optimal Stimulation Strategy

The equations for the amplitude response (31), (32) and (33) depend on the stimulation intensity V_{σ} at a population σ. It is implied, therefore, that the ‘population’ exists at some region in space and that V_{σ} should take into account the geometry of the electrode placement, how electric fields behave within brain tissue and the charges on a particular electrode. These ideas can be incorporated into an expression for the amplitude response.

Equations (31), (32) and (33) all involve summations over populations, with each term being the product of a weight w_{σ}, a stimulation intensity V_{σ} and some intrinsic response, denoted by Γ_{σ}. For example, in the case of Equation (31) Γ_{σ} would be

$${Γ}_{σ} = {\sum }_{m = 1}^{∞} {ρ}_{σ}^{m - 1} [ {α}_{m} sin [ ( m - 1 ) {ψ}_{σ} + ψ ] - {b}_{m} cos [ ( m - 1 ) {ψ}_{σ} + ψ ] ] - {\sum }_{m = 1}^{∞} {ρ}_{σ}^{m + 1} [ {α}_{m} sin [ ( m + 1 ) {ψ}_{σ} - ψ ] - {b}_{m} cos [ ( m + 1 ) {ψ}_{σ} - ψ ] ] , \qquad ( 46 )$$

Using this, a more compact expression for the amplitude response can be written using linear algebra notation, with r equal to the vector of responses and V equal to the vector of voltages at a population

$$\frac{d {ρ}_{stim}}{dt} = \frac{1}{2} ( {Γ}^{T} V ) \qquad ( 47 )$$

where the weights w_{σ} are now considered as part of the response. The amplitude response involves a ‘stimulation intensity’ V(t)—an abstract quantity which, intuitively, should not only depend on the charge characteristics at the electrode, but also the geometry of the electrode placement and the properties of the brain tissue. Taken altogether, the stimulation intensity is better interpreted as the voltage at a population, which can be expressed as a weighted superposition of charges at the electrodes

\tilde{D}q′=V (48)

As before, the elements of matrix \tilde{D} (of dimensions S×L) are coefficients which reflect the medium and geometry of the electrode-neuron system. It is worth noting here that Equations (45) and (48) can also be used to model systems where the stimulating and recording electrodes are different, since \tilde{D} is allowed to be different from D^{T}. Inserting (48) into (47) leads to an expression for the amplitude response in terms of the charges at the electrodes, i.e. the control variables.

$$\frac{d {ρ}_{stim}}{dt} = \frac{1}{2} {( {\tilde{D}}^{T} Γ )}^{T} {q}^{′} \qquad ( 49 )$$

The quantity (\tilde{D}^{T}Γ)^{T} is defined for each time step so that the optimisation becomes a problem of choosing q′ so as to minimise

$$\frac{d {ρ}_{stim}}{dt} .$$

Therefore, generating the plurality of stimulation signals comprises, for each of a plurality of time steps, choosing S18 the plurality of stimulation signals to maximally reduce the amplitude of the composite signal. Maximally reducing the amplitude of the composite signal can be achieved by minimising the rate of change in the amplitude of the composite signal. Ideally, the rate of change in the amplitude of the composite signal

$$( i . e . \frac{d {ρ}_{stim}}{dt} )$$

should be negative, with as large a magnitude as possible, such that the amplitude of the composite signal (and thereby the global order parameter representing synchrony of the neural oscillations) decreases over time. Minimising the rate of change in amplitude of the composite signal in this context means minimising at each time step, based on the instantaneous values of the relevant quantities. The rate of change in amplitude of the composite signal is calculated based on the composite signal and the plurality of population activities, as seen in the equations above.

Often, concern for tissue damage due to stimulation imposes a limit on how much charge can be delivered to a single or group of contact(s). To account for this and ensure feasibility, two constraints may be imposed. In some embodiments, the plurality of stimulation signals are chosen subject to a constraint on the charge applied by each electrode. This first constraint ensures the charge for a particular contact does not exceed some maximum value

0≤q′≤q′ₘₐₓ. (50)

A simple optimal solution (per time step) for Equations (49) and (50) can be found by setting the charge for the lth contact to q′ₘₐₓ if the lth component of (\tilde{D}^{T}Γ)^{T} is negative, i.e.:

$${q}_{l} = \{ \begin{matrix}{q}_{max}^{′} , {( {\tilde{D}}^{T} Γ )}_{l}^{T} < 0 \\ 0 , otherwise\end{matrix} \qquad ( 50 a )$$

In some embodiments, the plurality of stimulation signals are chosen subject to a constraint on the total charge density within a region of the subject, for example within a region of the brain in which the electrodes 7 are implanted or a region of the peripheral nervous system. This second constraint ensures the charge density within a region does not become dangerously high

Aq′≤q′ₘₐₓ. (51)

Here, for J groups of contacts, the constraint matrix A has dimension J×L and can be used to constrain the collective charges of the group. The J-dimensional vector q′ₘₐₓ specifies the maximum charge for a particular group of contacts. Equations (49), (50) and (51) are in the standard form for a linear program and are solvable in polynomial time.

We can write an analogous set of equations using estimated state variables (\tilde{ρ}_{σ}, \tilde{ψ}_{σ}, \tilde{ψ}) in terms of a set of ‘response parameters’ {C_{lσ}}. Letting

$${χ}_{l} ( t ) = [ {C}_{l 1} sin ( \tilde{ψ} ) + {C}_{l 2} cos ( \tilde{ψ} ) + {\sum }_{σ = 1}^{S} {C}_{l ( 2 + σ )} {\tilde{ρ}}_{σ} sin ( {\tilde{ψ}}_{σ} - \tilde{ψ} ) + {\sum }_{σ = 1}^{S} {C}_{l ( 2 + S + σ )} {\tilde{ρ}}_{σ} cos ( {\tilde{ψ}}_{σ} - \tilde{ψ} ) ] , \qquad ( 51 a )$$

the estimated response can be written as

$$\frac{d {\tilde{ρ}}_{stim}}{d t} = {\sum }_{l} {q}_{l} {χ}_{l} \qquad ( 51 b )$$

The estimated response as represented by Eq. (51a) and (51b) together has an analogous functional form to the expression for the actual response given in Eq. (33) above. The additional term in cos(\tilde{ψ}_{σ}−\tilde{ψ}) allows for a constant phase shift between the estimated and actual response. The ACR strategy can then be expressed compactly as

$${q}_{l} = \{ \begin{matrix}{q}_{max}^{′} , {χ}_{l} < 0 \\ 0 , otherwise\end{matrix} \qquad ( 51 c )$$

analogously to Eq. (50a) above.

One method for determining the response parameters involves delivering bursts of stimulation through each electrode indexed by l. The amplitude of the signal at the electrode is recorded at the beginning and end of each burst before calculating the change in amplitude Δ\acute{ρ}ₗⱼ for the j^{th} burst. This change due to stimulation can be expressed approximately as

$$Δ {\tilde{ρ}}_{l j} ≅ {\sum }_{n = 1} {( \frac{d {\tilde{ρ}}_{stim , l}}{d t} )}_{t = {t}_{j n}} Δ t \qquad ( 51 d )$$

During stimulation, the phases and amplitudes are recorded and the sinusoidal quantities from Equation (51a) are accumulated over time using (51d) leading to least squares equations for each electrode over J bursts which can be solved to obtain the response parameters for each of the electrodes

$$( \begin{matrix}{x}_{11} & {x}_{12} & {x}_{13} & … & {x}_{12 ( S + 1 )} \\ ⋮ & ⋱ & & & \\ ⋮ & & ⋱ & & \\ ⋮ & & & ⋱ & \\ {x}_{J 1} & & & & {x}_{J 2 ( S + 1 )}\end{matrix} ) ( \begin{matrix}{C}_{l 1} \\ {C}_{l 2} \\ {C}_{l 3} \\ ⋮ \\ {C}_{l 2 ( S + 1 )}\end{matrix} ) = ( \begin{matrix}Δ {\tilde{ρ}}_{l 1} \\ Δ {\tilde{ρ}}_{l 2} \\ Δ {\tilde{ρ}}_{l 3} \\ ⋮ \\ Δ {\tilde{ρ}}_{l J}\end{matrix} ) \qquad ( 51 e )$$

where the elements {x_{jσ}} are the sinusoidal variables from Equation (51a) accumulated over time of each burst according to (51d).

This fitting methodology is illustrated in FIG. 16. As discussed, ACR is calibrated using bursts of stimulation through each electrode to obtain the response parameters {C_{lσ}}. FIG. 16(a) shows how bursts of stimulation may be delivered in addition to the symptom signal. FIG. 16(b) shows that the amplitude is recorded at the beginning and end of the burst. At each stimulation point, the phases and amplitudes are recorded, which are later used to calculate the matrix elements {x_{jσ}} used to obtain the response coefficients according to Equation (51e).

Once the stimulation signals have been generated, they may be output for application to the subject. Application of the stimulation signals may be used for treatment of a variety of neurological conditions, including Parkinson's disease, epilepsy, obsessive compulsive disorder, and/or essential tremor For example, in the system 1 of FIG. 1, the processor 3 may output the stimulation signals to the electrical circuit 5 so that they can be provided to the electrodes 7.

### 5. NUMERICAL SIMULATIONS

The instantaneous response describes how the amplitude of a system should change as a function of its state variables, but did not take into account the dynamics of the system, such as the coupling which acts to resynchronise the oscillators and the effects of a finite number of oscillators—the latter leading to a breakdown in the underlying assumptions which lead to Equation (32). To better assess the real world performance of a particular stimulation strategy the time-averaged response is used, which requires simulating a system using equations (4), (27) and (26). Numerical simulations are used below to demonstrate that a coupled oscillator model is a plausible neural mechanism for generating tremor found in ET patients. On the basis of this, the activity of multiple neural populations is modelled using a set of oscillators and related to the pathological oscillations associated with symptom severity in ET and PD.

### 5.1. Simulated Systems

A system is defined in terms of its electrode-population configuration, dynamics and intrinsic response to stimulation Z(θ). To construct a particular system, the coordinates of S populations are randomly chosen such that they lie within a box of length L_{box}=10. Each population is assigned an electrode, which is placed dₙₒᵣₘ distance from the population. For a sufficiently large L_{box}, dₙₒᵣₘ can be used to characterise the system—a small dₙₒᵣₘ means the effects of stimulation are localised to a particular population and increasing dₙₒᵣₘ increasingly delocalises the effects of stimulation. For simplicity, a system consisting of S=4 populations and L=4 electrodes is considered. The analytical expressions for the response curves are for an infinite system, so N_{σ} should be large. For each population, the number of oscillators is chosen as N_{σ}=200 to satisfy this and to remain computationally feasible. A coulombic system is also assumed, where each electrode is able to simultaneously record and stimulate. In this case, the elements of D are given by

$${d}_{l σ} = \frac{{cw}_{σ}}{\vert {p}_{l}^{′} - {P}_{σ} \vert} . \qquad ( 52 )$$

The elements of matrix \tilde{D} are denoted \tilde{d}_{σl}, which can be related to D using the transpose

$${\tilde{d}}_{σ l} = \frac{{d}_{l σ}}{{cw}_{σ}} . \qquad ( 53 )$$

The dynamics of a system are determined by the parameters of the multipopulation Kuramoto model with an additional noise term

$$d {θ}_{σ n} = [ {ω}_{σ n} + {\sum }_{{σ}^{′} = 1}^{S} {w}_{{σ}^{′}} k {σσ}^{′} {ρ}_{{σ}^{′}} sin ( {ψ}_{{σ}^{′}} - {θ}_{σ n} ) + {V}_{σ} ( t ) {Z}_{σ} ( {θ}_{σ n} ) ] dt + \tilde{σ} N ( 0 , 1 ) \sqrt{dt} . \qquad ( 54 )$$

To simplify the testing, the basic parameters of (54) are fixed to those found from fitting to Patient 5. As previously mentioned, the natural frequencies {ω_{σn}} are sampled from a normal distribution. It is worth mentioning that this represents a greater test for the robustness of the expression for the amplitude response (32), which assumes a Lorentzian distribution for {ω_{σn}}.

The S×S coupling constant matrix can be simplified by focussing only on the diagonal and off-diagonal components, which are denoted k_{diag} and k_{offdiag} respectively. The value of k_{offdiag}=6, so that k_{diag} can be used to control the level of clustering for a particular configuration of oscillators—increasing k_{diag} leading to increasingly multimodal distributions of oscillators. The nPRC Z(θ) was also chosen according to parameters fitted to Patient 5, but the zeroth harmonic α₀ is allowed to vary.

### 5.2. Running the Simulation

To test each strategy a system is created according to the set of parameters {dₙₒᵣₘ, k_{diag}, α₀}, then a stimulation strategy is chosen from CR, phase-locked (PL), and the present novel method, ACR. The model is then used to describe how the neural population activity (and hence the symptom severity) is likely to change when DBS is applied through multiple contacts.

The implementation of PL stimulation illustrated here uses Equation (33), but neglects all the local terms, which is equivalent to setting ρ_{σ}=0. The time-shifted variant of CR neuromodulation [5, 22] is used in our testing. For a given electrode, stimulation is delivered in bursts of HF pulse trains. The stimulation pattern is time-shifted across each electrode indexed by l by

$${τ}_{l} = \frac{π}{2 \overline{ω}} ( l - 1 ) , \qquad ( 55 )$$

where ω is the mean of the natural frequencies (≈4.2 Hz). The number of bursts per second, the burst frequency f_{burst}, was chosen to be equal to ω/2π and the HF pulse train frequency fₜᵣₐᵢₙ was chosen to be 130 Hz. The width of each burst t_{burst} was chosen to be 0.1 seconds. Tass et al originally tested CR on a homogeneously coupled system with s_{ω}=0 [5]. The present implementation was tested and reproduces these results by constructing a simple homogeneously coupled system according to the parameters of Patient 5 given in Table 2, but with \tilde{σ}=0, s_{ω}=0 and the parameters of Z(θ) scaled by a factor of 10. The simulation parameters were chosen according to Table 3.

The desynchronising effects of CR neuromodulation on this system are shown in FIG. 9. FIG. 9 shows the output from numerical simulations showing the effects of Coordinated Reset (CR). Stimulation was turned on at t=20 seconds. The top panel of (a) shows the model output for a system simulated according to Equations (54) and (24). The bottom panel of (a) shows the stimulation delivered as a function of time, taken to be the average of the charges across the contacts. The bottom panel of (b) shows the stimulation across each contact, with the corresponding model output provided in the top panel. The results in FIG. 9 reproduce the results of Tass et al [5].

The maximum charge for an electrode q′ₘₐₓ is chosen so that, for a given system, the maximum perturbation to a single oscillator is dθₘₐₓ=0.2πrad. For the testing, ACR is not constrained using Equation (51), which leads to trivial optimal solutions to the linear program (49) and (50), where the charge for the lth contact is set to q′ₘₐₓ if the lth component of (\tilde{D}^{T}Γ)^{T} is negative. ACR was tested at 3 maximum stimulation frequencies: 5 Hz, 50 Hz and 130 Hz. It should be noted that because ACR is applied according to a feedback signal (i.e. the sensor signals from the sensors), the stimulation frequency can vary, but the maximum frequency introduces an upper limit on the allowed frequencies.

The maximum stimulation frequency for PL was fixed at 130 Hz. Equation (54) was then integrated using Euler's method with a time step of t=0.04 seconds and simulated for T=2 seconds. The PL and ACR strategies were applied according to phases and amplitudes obtained directly from the simulation.

During a simulation, a stimulation pulse is calculated as the average of the charges q′(t) across the L electrodes. Two quantities are calculated after each simulation: the time-averaged value of ρ, ρ and the total of all stimulation pulses E delivered. The former is indicative of the efficacy of the strategy while the latter is related to the total energy consumption of a strategy, which can be used to gauge efficiency. For each set of parameters, the simulations are repeated over 24 trials, with a new electrode-population configuration being generated according to dₙₒᵣₘ for each trial. The parameters dₙₒᵣₘ and k_{diag} were chosen within the range dₙₒᵣₘ∈[0.1,6] and k_{diag}∈[5,150]. Example output from these simulations, showing the effects of applying ACR, is provided in FIG. 10. FIG. 10 shows the output from numerical simulations showing the effects of Adaptive Coordinated Reset (ACR). Stimulation was turned on at t=20 seconds. The top panel of (a) shows the model output for a system simulated according to Equations (54) and (24). The bottom panel of (a) shows the stimulation delivered as a function of time, taken to be the average of the charges across the contacts. The bottom panel of (b) shows the stimulation across each contact, with the corresponding model output provided in the top panel.

When compared to FIG. 9, it is clear that the stimulation pattern from ACR is significantly different from that produced by CR, with the latter pattern being simply time-shifted across electrodes. The stimulation pattern from ACR allows for the possibility that multiple electrodes may be stimulated simultaneously. A summary of the parameters used in these simulations is provided in Tables 3 and 4.

TABLE 3
Summary of fixed parameters used in the simulations.

| Parameter | Value | Description |
|---|---|---|
| Δt | 0.04 | Integration time step |
| T | 2 | Simulation time |
| L_{box} | 10 | Box length |
| L | 4 | Number of electrodes |
| S | 4 | Number of populations |
| N_{σ} | 200 | Number of oscillators per population |
| k_{offdiag} | 6 | Off-diagonal of coupling constant matrix |
| dθₘₐₓ | 0.2π | Maximum angle moved per stimulation pulse |
| nₜᵣᵢₐₗₛ | 24 | Number of trials |
| fₘₐₓ | 130 | Maximum frequency for PL stimulation |
| f_{burst} | 4.2 | CR burst frequency |
| fₜᵣₐᵢₙ | 130 | CR HF train frequency |
| t_{burst} | 0.1 | CR burst time |
| ω̅/2π | 4.2 | Mean oscillator frequency |
| s_{ω}/2π | 0.7 | Standard deviation of oscillator frequency |

TABLE 4
Summary of variable parameters used in the simulations.
Each parameter was chosen in the range [Min, Max]
using a uniform grid of spacing (Max-Min)/Ngrid.

| Parameter | Min | Max | N_{grid} | Description |
|---|---|---|---|---|
| dₙₒᵣₘ | 0.1 | 6 | 5 | Distance from population to electrode |
| k_{diag} | 5 | 150 | 5 | Diagonal of coupling constant matrix |
| a₀ | 0 | 1.7 | 4 | Zeroth harmonic of Z(θ) |

### 5.3. Results

The results demonstrate a strategy for providing DBS through multiple contacts in order to maximally desynchronise neural activity, and thereby reduce symptom severity. Numerical simulation and parameters fitted to ET patients are used to compare this method to other known methods, namely phase-locked stimulation and coordinated reset. The numerical simulations demonstrate that the present method has the potential to be both more effective and efficient than existing methods.

ACR was tested at maximum frequencies of 130 Hz, 50 Hz and 5 Hz. The maximum frequency of PL was fixed at 130 Hz. The rise in p shown between k_{diag}=50 and k_{diag}=70 is indicative of a bifurcation, which is typical in Kuramoto systems [16]. Significant improvements with ACR over PL and CR are observed in simulations when stimulation is delivered at higher frequencies and when α₀ is non-negligible. The utility of ACR over other methods is also shown to be greatest when k_{diag} is larger, which corresponds to larger local amplitudes ρ_{σ} and increased clustering.

The efficacy of CR can also be seen to improve with systems with larger α₀. FIG. 11 shows plots for the average amplitude ρ of a simulated Kuramoto system (i.e. ρ averaged over all trials and all values of dₙₒᵣₘ for a particular value of k_{diag} and zeroth harmonic α₀) for different stimulation strategies. The stimulation strategies used were no stimulation (no stim), Adaptive Coordinated Reset (ACR), phase-locked (PL) and Coordinated Reset (CR). The maximum stimulation frequency used for ACR and PL is also given in the legend. Dashed lines are for the ACR method. Each sub-plot shows a set of simulations performed with a particular zeroth harmonic of the nPRC α₀.

With α₀=0, CR and no stimulation are shown to be equally effective. The results shown in FIG. 11 are in contrast with those shown in FIG. 9, indicating that the efficacy of CR is sensitive to the parameters \tilde{σ} and s_{ω} in addition to the scaling of Z(θ). For systems where α₀≈0, PL and ACR are found to be equally as effective, as predicted. The efficacy of ACR at 130 Hz is greater than at other frequencies, but with more energy usage than PL and similar energy usage to CR, as shown in FIG. 12. FIG. 12 shows the average energy used by the different stimulation strategies on a simulated Kuramoto system with a coupling constant k_{diag}, namely no stimulation (no stim), Adaptive Coordinated Reset (ACR), phase-locked (PL) and Coordinated Reset (CR). The maximum stimulation frequency used for ACR and PL is given in the legend. Dashed lines are for the ACR method. Each sub plot shows a set of simulations performed with a particular zeroth harmonic of the nPRC α₀. ACR at 50 Hz is found to have good efficacy for α₀>0 but with significantly less energy usage than PL and CR. ACR with low frequency stimulation at 5 Hz (or approximately the tremor frequency) is predicted to have little to no effect on all the systems tested.

As previously mentioned, the utility of ACR is expected to be greatest for those systems described by type I nPRCs, as the amplitude response curve then depends explicitly on non-negligible terms involving population quantities. Equation (32) shows that when α₀=0, the terms involving population quantities depend on second harmonics 2ψ_{σ}. These terms carry a factor of ρ_{σ}² and hence are likely to be negligible, except when ρ_{σ} is reasonably large. To investigate these effects, systems with α₀=0 are simulated, and a larger stimulation intensity q′ₘₐₓ used. By comparing ACR to PL, the impact of these second harmonic terms can be ascertained.

A comparison of the efficacy of ACR with PL and CR, is shown in FIG. 13 for different stimulation amplitudes q′ₘₐₓ and with α₀=0. Increasing stimulation amplitude can be seen to improve the efficacy of all the strategies tested, but is most noticeable for ACR and PL. Higher stimulation amplitudes also seem to be particularly beneficial for those systems with larger k_{diag}. ACR can be seen to perform better than CR in all cases. FIG. 13 shows the average amplitude of a simulated Kuramoto system with a coupling constant k_{diag} for different stimulation strategies: no stimulation (no stim), Adaptive Coordinated Reset (ACR), phase-locked (PL) and Coordinated Reset (CR). During these simulations, the zeroth harmonic α₀ of the nPRC Z(θ) was fixed to zero. Increasing dθ_max leading to larger stimulation amplitudes q′ₘₐₓ shows the effect of the second harmonic term in the amplitude response given by Equation (32). FIG. 13 shows the ACR method to be consistently more effective than PL at higher k_{diag}, although the difference is marginal. This is indicative of the aforementioned effects of second harmonic terms in (32). Taken altogether, the efficacy of ACR is predicted to be similar to PL stimulation for those systems where α₀≈0.

The mathematical description of ACR predicts that the effectiveness of multi-contact stimulation is largely dependent on the form of the nPRC and in particular on the zeroth harmonic α₀, which is related to whether the nPRC is type I or type II. The dependency of the amplitude response on the local quantities of population a becomes less at increasingly lower local amplitudes ρ_{σ}, but the effects of stimulation are, in general, explicitly dependent on the state of the neuronal system and providing stimulation without knowledge of this state is likely to be suboptimal. For type II systems, where |α₀| is small, stimulation on the basis of local quantities is unlikely to be beneficial. Therefore, depending on the form of the phase response curve obtained using the composite signal, it can be determined whether or not it is worthwhile using the multi-channel LFP data in addition to the composite signal in a closed-loop DBS strategy.

The ACR method assumes an underlying system of phase oscillators, which can be divided into small populations with the distribution of oscillators in each population satisfying the ansatz of Ott and Antonsen [16]. Equation (44) links the state to measurable quantities from the electrode and is of the form modelled by ICA. The ability to resolve the state and parameters of the system does not factor directly into the results presented in Section 5 since both the state and the parameters were taken directly from the simulation.

Therefore, the results herein can be taken as an upper bound on performance.

### 5A. Further Numerical Testing

Further testing of ACR was performed according to the aforementioned methods, across a variety of test systems. The simulations can be summarised as follows:
(1) Simulate an infinite multipopulation Kuramoto system to obtain simulated electrode measurements {Vₗ}.
(2) Use ICA to estimate {f_{σ}} from {Vₗ} and obtain the inverse matrix elements {g_{σl}}.
(3) Construct an estimated symptom signal \tilde{f}(t) according to (45a) by choosing the weights {\tilde{w}_{σ}} to best correlate \tilde{f}(t) with the symptom.
(4) Obtain the response parameters {C_{lσ}} using the linear least squares model.
(5) Using the inverse matrix elements {g_{σl}} and phase/amplitude tracking, apply ACR according to Equation (51c).

A schematic illustration of closed-loop DBS with ACR is shown in FIG. 17. The Kuramoto model produces simulated electrode recordings which are passed through an interface (grey box). The interface consists of inverse matrix elements found through ICA and a phase/amplitude tracking algorithm. The inverse matrix elements transform the electrode recordings into estimations for the population activities. The estimated population activities are then passed to the phase/amplitude tracking algorithm to estimate the state. The estimated state is then used in the ACR equations to determine whether stimulation should be delivered. The ACR equations here use parameters estimated from the data.

FIG. 18 shows visualisations of the 3 systems considered in the further testing. Each system consists of 4 neural populations, 4 electrodes, and 4 sensors. In this testing, the electrodes and sensors are separate, and so are referred to as stimulating electrodes and recording electrodes respectively. The geometry of the system is characterised by a ‘configuration parameter’ dₙₒᵣₘ. As discussed above in relation to FIG. 8, for larger values of dₙₒᵣₘ, the populations are more dispersed relative to the electrodes. In the scenario of FIG. 18, stimulation from electrodes may affect multiple populations.

Simulated electrode recordings due to the activities of each population are shown in FIG. 19. Each plot shows individual channel measurements of duration T_{ICA}=2000 seconds for each of the 4 recording electrodes which serve as inputs for ACR. ICA is applied to this data to produce estimates of the population activities shown in FIG. 20. The theoretical data has been overlaid for comparison and shows good agreement with the output from ICA. The symptom signal was calculated according to (24) with {w_{σ}}=0.25.

The estimated symptom signal was calculated according to (45a) by using the estimated activities {\tilde{f}_{σ}} and optimising the estimated weights {\tilde{w}_{σ}} to produce a signal which closely matches the symptom signal. The total simulation time used in the parameter estimation was T_{PFIT}=3840 seconds.

FIG. 21 shows the symptom signals for a variety of systems after applying ACR using estimated states and parameters. Testing was performed over a variety of systems according to the neural response α₀ and system geometry dₙₒᵣₘ. Each plot shows the symptom signal (Osc) and the stimulation triggers (Stim). The stimulation in each case was turned on at t=15 seconds. In each case, applying ACD can be seen to significantly desynchronise the systems considered.

### 6. EXPERIMENTAL DATA

Cagnan et al [7] studied phase-locked DBS delivered according to the tremor in ET patients. Data was collected from 6 ET patients and 3 dystonic tremor patients. All patients gave their informed consent to take part in the study, which was approved by the local ethics committee in accordance with the Declaration of Helsinki. The data from this study can be obtained through an online repository [19].

Duchet et al [18] defined a criterion for assessing significance in the averaged ARCs and PRCs from the study of Cagnan et al. In their study, a patient is considered to have a significant response if both the ARC and PRC are found to be significant either according to an ANOVA test or cosine model F-test. Using this, they deemed 3 out of the 6 ET patients to have a significant response curve. The analysis herein is restricted to these 3 patients, who are referred to as patients 1, 5 and 6, as in the original study. The tremor data was filtered using a non-causal Butterworth filter of order 2 with cut-off frequencies at ±2 Hz around the tremor frequency. Stimulation was delivered over a set of trials (typically 9), with each trial consisting of 12 blocks of 5 second phase-locked stimulation at a randomly chosen phase from a set of 12. Each block of phase-locked stimulation was also separated by a 1 second interblock of no stimulation. The envelope amplitude and instantaneous phase were calculated using the Hilbert transform.

As an example, the data for Patient 1 is shown in FIG. 14. Tremor oscillations are shown in the top panels. The bottom panels shows the stimulation triggers. (a) shows the entirety of the dataset consisting of stimulation provided over 9 trials. (b) shows a single trial which consists of 5 seconds of phase-locked stimulation over 12 phases.

From this, the characteristics identified as desirable for the model to reproduce are: the frequency spectrum of the data, the bursts of oscillations and the sustained periods of low envelope amplitude. In addition, the model preferably reproduces a given patient's response to stimulation, as characterised by the averaged PRC.

### REFERENCES

[1] Yousif N, Mace M, Pavese N, Borisyuk R, Nandi D, Bain P. A network model of local field potential activity in essential tremor and the impact of deep brain stimulation. PLoS computational biology. 2017; 13(1):e1005326.
[2] Little S, Pogosyan A, Neal S, Zavala B, Zrinzo L, Hariz M, et al. Adaptive deep brain stimulation in advanced Parkinson disease. Annals of neurology. 2013; 74(3):449-457. 552
[3] Hua S, Lenz F, Zirh T, Reich S, Dougherty P M. Thalamic neuronal activity correlated with essential tremor. Journal of Neurology, Neurosurgery &Psychiatry. 1998; 64(2):273-276.
[4] Koller W, Pahwa R, Busenbark K, Hubble J, Wilkinson S, Lang A, et al. High-frequency unilateral thalamic stimulation in the treatment of essential and parkinsonian tremor. Annals of Neurology: Ocial Journal of the American Neurological Association and the Child Neurology Society. 1997; 42(3):292-299. 558
[5] Tass P A. A model of desynchronizing deep brain stimulation with a demand-controlled coordinated reset of neural subpopulations. Biological cybernetics. 2003; 89(2):81-88. 560
[6] Peles O, Werner-Reiss U, Bergman H, Israel Z, Vaadia E. Phase-Specific Microstimulation Differentially Modulates Beta Oscillations and Affects Behavior. Cell Reports. 2020; 30(8):2555-2566.
[7] Cagnan H, Pedrosa D, Little S, Pogosyan A, Cheeran B, Aziz T, et al. Stimulating at the right time: phase-specific deep brain stimulation. Brain. 2016; 140(1):132-145. 567
[8] Weerasinghe G, Duchet B, Cagnan H, Brown P, Bick C, Bogacz R. Predicting the effects of deep brain stimulation using a reduced coupled oscillator model. PLoS computational biology. 2019; 15(8):e1006575.
[9] Brown E, Moehlis J, Holmes P. On the phase reduction and response dynamics of neural oscillator populations. Neural computation. 2004; 16(4):673-715.
[10] Hansel D, Mato G, Meunier C. Synchrony in excitatory neural networks. Neural computation. 1995; 7(2):307-337.
[11] Schultheiss N W, Edgerton J R, Jaeger D. Phase response curve analysis of a full morphological globus pallidus neuron model reveals distinct perisomatic and dendritic modes of synaptic integration. Journal of Neuroscience. 2010; 30(7):2767-2782. 577
[12] Cagnan H, Brittain J S, Little S, Foltynie T, Limousin P, Zrinzo L, et al. Phase dependent modulation of tremor amplitude in essential tremor through thalamic stimulation. Brain. 2013; 136(10):3062-3075.
[13] Holt A B, Kormann E, Gulberti A, Poetter-Nerger M, McNamara C G, Cagnan H, et al. Phase-dependent suppression of beta oscillations in Parkinson's disease patients. Journal of Neuroscience. 2019; 39(6):1119-1134.
[14] Stiefel K M, Gutkin B S, Sejnowski T J. Cholinergic neuromodulation changes phase response curve shape and type in cortical pyramidal neurons. PloS one. 2008; 3(12):e3947.
[15] Fong R, Russell J, Weerasinghe G, Bogacz R. Kuramoto Model Simulation; 2018. University of Oxford, doi:10.5287/bodleian:6qJdGNDPj, available at: https://data.mrc.ox.ac.uk/data-set/kuramoto.
[16] Ott E, Antonsen T M. Low dimensional behavior of large systems of globally coupled oscillators. Chaos: An Interdisciplinary Journal of Nonlinear Science. 2008; 18(3):037113.
[17] Bick C, Goodfellow M, Laing C R, Martens E A. Understanding the dynamics of biological and neural oscillator networks through exact mean-field reductions: a review. The Journal of Mathematical Neuroscience. 2020; 10(1):1-43.
[18] Duchet B, Weerasinghe G, Cagnan H, Brown P, Bick C, Bogacz R. Phase-dependence of response curves to deep brain stimulation and their relationship: from essential tremor patient data to a Wilson-Cowan model. The Journal of Mathematical Neuroscience. 2020; 10(1):1-39.
[19] Cagnan H, Weerasinghe G, Brown P. Tremor data measured from essential tremor patients subjected to phase-locked deep brain stimulation; 2019. University of Oxford, doi:10.5287/bodleian:xq24eN2 Km, available at: https://data.mrc.ox.ac.uk/data-set/tremor-data-measured-essential-tremor-patients-subjected-phase-locked-deep-brain.
[20] Goetz C G, Tilley B C, Shaftman S R, Stebbins G T, Fahn S, Martinez-Martin P, et al. Movement Disorder Society-sponsored revision of the Unified Parkinson's Disease Rating Scale (MDS-UPDRS): scale presentation and clinimetric testing results. Movement disorders: official journal of the Movement Disorder Society. 2008; 23(15):2129-2170.
[21] Hagen E, Naess S, Ness T V, Einevoll G T. Multimodal modeling of neural network activity: computing LFP, ECoG, EEG, and MEG signals with LFPy 2.0. Frontiers in neuroinformatics. 2018; 12:92.
[22] Tass P A, Majtanik M. Long-term anti-kindling effects of desynchronizing brain stimulation: a theoretical study. Biological cybernetics. 2006; 94(1):58-66.
[23] Hofmanis J, Ruiz R A S, Caspary O, Ranta R, Louis-Dorr V. Extraction of Deep Brain Stimulation (DBS) source in SEEG using EMD and ICA. In: 2011 Annual International Conference of the IEEE Engineering in Medicine and Biology Society. IEEE; 2011. p. 834-837.
[24] Abbasi O, Hirschmann J, Schmitz G, Schnitzler A, Butz M. Rejecting deep brain stimulation artefacts from MEG data using ICA and mutual information. Journal of neuroscience methods. 2016; 268:131-141.
[25] Oswal A, Jha A, Neal S, Reid A, Bradbury D, Aston P, et al. Analysis of simultaneous MEG and intracranial LFP recordings during Deep Brain Stimulation: a protocol and experimental validation. Journal of neuroscience methods. 2016; 261:29-46.
[26] Lagarias J C, Reeds J A, Wright M H, Wright P E. Convergence properties of the Nelder-Mead simplex method in low dimensions. SIAM Journal on optimization. 1998; 9(1):112-147.

1. A method of generating deep brain stimulation signals, the method comprising:
receiving a plurality of sensor signals from a corresponding plurality of sensors on or in a subject; and
using the received sensor signals to generate a plurality of stimulation signals for application at a corresponding plurality of target sites in the brain of the subject.

2. The method of claim 1, wherein the generating of the plurality of stimulation signals comprises using a model of the response of neurons in the subject to the stimulation signals that models neural tissue as a plurality of coupled populations of neurons.

3. A method of generating stimulation signals, the method comprising:
receiving a plurality of sensor signals from a corresponding plurality of sensors on or in a subject; and
using the received sensor signals to generate a plurality of stimulation signals for application at a corresponding plurality of target sites on or in the subject using a model of the response of neurons in the subject to the stimulation signals that models neural tissue as a plurality of coupled populations of neurons.

4. The method of claim 3, wherein generating the plurality of stimulation signals comprises determining a population activity for each population of neurons and determining the amplitude and phase of each population activity, the population activity of each population being a measure of neural activity among neurons in that population.

5. The method of claim 4, wherein determining the population activities comprises applying independent component analysis to the sensor signals.

6. The method of claim 4, wherein generating the plurality of stimulation signals further comprises determining a composite signal using a weighted combination of the population activities, and determining the amplitude and phase of the composite signal.

7. The method of claim 6, wherein generating the plurality of stimulation signals further comprises, for each of a plurality of time steps, choosing the plurality of stimulation signals to maximally reduce the amplitude of the composite signal over the time step.

8. The method of claim 7, wherein maximally reducing the amplitude of the composite signal comprises, for each time step, calculating the rate of change in amplitude of the composite signal using the composite signal and the plurality of population activities.

9. The method of claim 7, wherein the plurality of stimulation signals are chosen subject to a constraint on the total charge density within a region of the subject.

10. The method of claim 7, wherein the plurality of stimulation signals are chosen subject to a constraint on the charge applied by each electrode.

11. The method of claim 6, wherein the weights in the weighted combination are such that the amplitude of the composite signal is correlated to a measure of severity of a symptom of the subject.

12. The method of claim 11, wherein the amplitude of the composite signal is proportional to the measure of severity.

13. The method of claim 3, wherein the model models each population of neurons as a plurality of coupled oscillators.

14. The method of claim 13, wherein the plurality of coupled oscillators are a plurality of coupled Kuramoto oscillators.

15. The method of claim 13, wherein the model models the response of the neurons to the stimulation signals as being dependent on the phase of oscillations of the neurons.

16. The method of claim 3, wherein the plurality of sensor signals represent electrical activity in the subject.

17. The method of claim 16, wherein the electrical activity is a local field potential produced by neurons in a region of the brain of the subject.

18. The method of claim 3, wherein the sensors are inertial sensors, and the plurality of sensor signals represent movement of the subject.

19. The method of claim 3, wherein the stimulation signals are deep brain stimulation signals, and the target sites are in the brain of the subject.

20. The method of claim 3, wherein the stimulation signals comprise a plurality of pulses.

21. The method of claim 3, wherein the stimulation signals have a carrier frequency of at least 20 Hz and/or at most 250 Hz.

22. The method of claim 3, wherein the application of the stimulation signals is used for treatment of Parkinson's disease, epilepsy, obsessive compulsive disorder, and/or essential tremor.

23. The method of claim 3, further comprising applying the plurality of stimulation signals at the corresponding plurality of target sites.

24. A system for applying stimulation signals, the system comprising:
a processor configured to generate a plurality of stimulation signals according to the method of claim 3; and
an electrical circuit configured to provide the plurality of stimulation signals to a corresponding plurality of electrodes for application at the plurality of target sites.

25. The system of claim 24, wherein the electrical circuit comprises the plurality of electrodes.

26. The system of claim 24, wherein the plurality of electrodes are configured to be implanted into the brain of the subject.

27. The system of claim 24, further comprising a plurality of sensors configured to be placed on or in the subject, the sensors configured to generate the plurality of sensor signals, and transmit the sensor signals to the processor.

28. The system of claim 27, wherein the sensors are configured to be implanted into the brain of the subject, and the plurality of sensor signals represent electrical activity in the brain of the subject.

29. The system of claim 27, wherein the plurality of sensors are the plurality of electrodes.

30. The system of claim 27, wherein the sensors are inertial sensors, and the plurality of sensor signals are inertial signals representing movement of the subject.

31. A computer program comprising instructions which, when the program is executed by a computer, cause the computer to carry out the method of claim 3.
